(19) United States
(12) Patent Application Publication
(10) Pub. No.: US 2017/0198354 A1
(43) Pub. Date: Jul. 13, 2017

(54) METHODS OF GLUCOCORTICOID THERAPY AND DETERMINING RESPONSIVENESS THEREOF

(71) Applicant: YISSUM RESEARCH DEVELOPMENT COMPANY OF THE HEBREW UNIVERSITY OF JERUSALEM LTD., Jerusalem (IL)

(72) Inventors: Eitan YEFENOF, Jerusalem (IL); Shlomit KFIR-ERENFELD, Jerusalem (IL)

(21) Appl. No.: 15/314,153

(22) PCT Filed: May 27, 2015
(86) PCT No.: PCT/IL2015/050552

§ 371 Date: Nov. 28, 2016

Related U.S. Application Data
(60) Provisional application No. 62/003,622, filed on May 28, 2014.

(51) Int. Cl. C12Q 1/68; A61K 31/713; A61K 31/573; C12N 15/113

(52) U.S. Cl. CPC C12Q 1/6886; C12N 15/113; A61K 31/713; A61K 31/573; C12N 2310/141; C12N 2320/31; C12Q 2600/178; C12Q 2600/106; C12Q 2600/158

(57) ABSTRACT

The invention provides one or more miRNAs, including but not limited miR-103, miR-30d, and miR-30e, for predicting the response of cancer cells to glucorticoids treatment. The invention further provides therapeutic uses of said miRNA molecules for increasing cells sensitivity to glucorticoids induced cell apoptosis.

### FIELD OF INVENTION

The present invention is directed to, inter alia, compositions and methods for identifying subjects suitable for glucocorticoids treatment. The invention further relates to compositions and methods for improving a therapeutic response to glucocorticoids treatment.

### BACKGROUND OF THE INVENTION

Glucocorticoids (GCs) are widely used in the therapy of hematopoietic malignancies of the lymphoid lineage due to their ability to induce apoptosis of lymphoid cells. The main hematopoietic cancer types that respond well to GC therapy include T acute lymphoblastic leukemia (T-ALL), chronic B lymphocytic leukemia (CLL), multiple myeloma (MM), Hodgkin's lymphoma (HL), and non-Hodgkin's lymphoma (NHL). A major impediment in GC therapy is the gradual acquisition of resistance to the drug following repeated treatments. Furthermore, some patients are a priori resistant to GC therapy.

Acute lymphoblastic leukemia (ALL), as a non-limiting example, afflicts mostly children with a peak incidence at 2-5 years of age. The disease is characterized by accumulation of abnormal immature lymphoblasts in the bone marrow (BM) which damage the production of other normal cells such as lymphocytes, red blood cells, granulocytes and platelets. In contrast to normal cells, immature ALL cells also exit the BM into the blood. Poor response to a 7-day monotherapy with the GC analogue prednisone (PRED) is one of the strongest predictors of adverse outcomes in the treatment of pediatric ALL.

Inflammation is normally a localized, protective response to trauma or microbial invasion that destroys, dilutes, or walls-off the injurious agent and the injured tissue. Excessive inflammation caused by abnormal recognition of host tissue as foreign, or prolongation of the inflammatory process, may lead to inflammatory diseases including but not limited to asthma, atherosclerosis, rheumatic fever and rheumatic diseases such as systemic lupus erythematosus and rheumatoid arthritis. Diseases characterized by inflammation are significant causes of morbidity and mortality in humans.

As a non-limiting example, current methods of treating asthma involve the use of corticosteroids, β2-agonists or leukotriene antagonists. Although asthma has been treated by these methods for several years, a significant fraction of asthma patients are resistant to treatment. As there are risks associated with methods for treating asthma, identification of patients that will be responsive to treatment is important.

There is an unmet need for compositions and methods capable of distinguishing between GC resistant and GC-sensitive patients. There is a further unmet need for therapeutic compositions and method for improving a therapeutic response to GC treatment, such as, by conferring GC-sensitivity on otherwise GC-resistant cells.

### SUMMARY OF THE INVENTION

The present invention provides methods, compositions and kits for identifying subjects suitable for glucocorticoid (GC) treatment. The invention further provides methods, compositions and kits for treating subjects with GC, including but not limited to, GC resistant subjects.

The present invention is based, in part, on the finding that expression levels of specific miRNAs including, but not limited to, miR-103, miR-30e, miR-30d, miR-181a*, miR-15b* and miR-21, serve as a diagnostic tool for predicting responsiveness of patient to GC treatment.

The present invention is also based, in part, on the unexpected finding that manipulating specific miR expression (e.g., of miR-103) results in sensitizing GC-resistant cancer cells to GC treatment.

According to one aspect, there is provided a method for predicting responsiveness of a subject to GC treatment, the method comprising determining the expression level of one or more miRNAs selected from the group consisting of: miR-103, miR-30e, miR-30d, miR-181a*, miR-15b* and miR-21, in a sample obtained from the subject, wherein modulation of expression levels of said one or more miRNAs compared to control indicates that the subject will be responsive to GC treatment.

In one embodiment, the method comprises determining the expression level of miR-103, wherein increased expression levels of miR-103 compared to control indicates that said subject will be responsive to GC treatment.

In another embodiment, the method comprises determining the expression level a plurality of miRNAs selected from the group consisting of: miR-103, miR-30e, miR-30d, miR-181a*, miR-15b* and miR-21, wherein modulation of expression levels of said plurality of miRNAs compared to control indicates that the subject will be responsive to GC treatment. In another embodiment, the method further comprises determining the expression level of at least one miRNA selected from miR-15b, miR-16, miR-181a, let-7f, miR-186, miR-223, miR-103*, miR-20a, miR-92a, miR-17, miR-30e*, miR-19b, miR-18a and miR-19a, wherein modulation of expression levels of at least one miRNA compared to control indicates that the subject will be responsive to GC treatment.

According to another aspect, there is provided a method for treating a subject with GC, the method comprising:
i. determining the expression levels of a one or more miRNA selected from the group consisting of miR-103, miR-30e, miR-30d, miR-181a*, miR-15b* and miR-21, in a sample obtained from a subject, wherein modulation of expression levels of said one or more miRNA compared to control indicates that the subject is susceptible for GC treatment; and
ii. administering a therapeutically effective amount of GC to the susceptible subject. Thereby treating said subject with GC.

In another embodiment, step (i) comprises determining the expression level miR-103, wherein increased expression levels of miR-103 compared to control indicates that said subject is susceptible for GC treatment.

In another embodiment, step (i) further comprises determining the expression level a plurality of miRNAs selected from the group consisting of: miR-103, miR-30e and miR-30d, miR-30e, miR-30d, miR-181a*, miR-15b* and miR-21, wherein modulation of expression levels of said plurality of miRNAs compared to control indicates that said subject is susceptible for GC treatment.

In another embodiment, step (i) further comprises determining the expression level of at least one additional miRNA selected from the group consisting of miR-15b, miR-16, miR-181a, has-let-7f, miR-186, miR-223, miR-103*, miR-20a, miR-92a, miR-17, miR-30e*, miR-19b, miR-18a, miR-19a, wherein modulation of expression levels of said plurality of miRNAs compared to control indicates that said subject is susceptible for GC treatment.

According to some embodiments of the invention, said modulation of expression is increased expression. In some embodiments, said modulation of expression is statistically significant increased expression. In another embodiment, increased expression of one or more miRNAs selected from the group consisting of miR-103, miR-30e, miR-30d, miR-15b*, miR-21, miR-15b, miR-16, miR-181a, let-7f, miR-186 and miR-223 indicates that the subject will be responsive to GC treatment.

According to some embodiments of the invention, said modulation of expression is decreased expression. In some embodiments, said modulation of expression is statistically significant decreased expression. In another embodiment, decreased expression of one or more miRNAs selected from the group consisting of miR-181a*, miR-103*, miR-20a, miR-92a, miR-17, miR30e*, miR-19b, miR-18a and miR-19a indicates that the subject will be responsive to GC treatment.

According to some embodiments of the methods, compositions and kits of the invention, said subject is afflicted with a disease typically treated with GCs. According to some embodiments of the methods, compositions and kits of the invention, said subject is afflicted with cancer. According to some embodiments of the methods, compositions and kits of the invention, said subject is afflicted with a cancer typically treated with GCs, including but not limited to hematopoietic cancer, osteosarcoma or small cell lung cancer. According to some embodiments, said cancer is hematopoietic cancer. In another embodiment, said cancer is primary cancer. In another embodiment, said cancer is relapsed cancer.

According to some embodiments, said sample is selected from blood, plasma and serum. According to another embodiment, said sample is peripheral blood lymphoblasts (PBLs).

According to another aspect, there is provided a kit comprising reagents adapted to specifically determine the expression level of a plurality of miRNAs selected from the group consisting of: miR-103, miR-30e, miR-30d, miR-181a*, miR-15b* and miR-21.

According to another aspect, there is provided a kit for predicting susceptibility of a subject to GC therapy, the kit comprising reagents adapted to specifically determine the expression level of one or more miRNAs selected from the group consisting of: miR-103, miR-30e, miR-30d, miR-181a*, miR-15b* and miR-21.

In another embodiment, the kit comprises reagents adapted to determine specifically the expression level of miR-103. In another embodiment, the kit comprises reagents adapted to specifically determine the expression level of a plurality of miRNAs selected from the group consisting of: miR-103, miR-30e, miR-30d, miR-181a*, miR-15b* and miR-21. In another embodiment, the kit further comprises reagents adapted to specifically determine the expression level of at least one additional miRNA selected from miR-15b, miR-16, miR-181a, has-let-7f, miR-186, miR-223, miR-103*, miR-20a, miR-92a, miR-17, miR-30e*, miR-19b, miR-18a and miR-19a.

In another embodiment, said reagents are selected from miRNA hybridization or amplification reagents, and one or more miRNAs-specific probe or amplification primer.

In another embodiment, said kit further comprises means for obtaining a blood sample. In another embodiment, said kit is identified (e.g., by incorporation of a label) for predicting susceptibility of a subject to GC therapy.

According the another aspect, there is provided a method for sensitizing cells of a subject in need thereof to GC therapy, the method comprising administering to the subject a pharmaceutical composition comprising one or more miRNAs selected from the group consisting of miR-103, miR-30e, miR-30d, miR-15b* and miR-21, and a pharmaceutically acceptable carrier. In another embodiment, the composition further comprises one or more miRNAs selected from the group consisting of miR-15b, miR-16, miR-181a, let-7f, miR-186 and miR-223. In another embodiment, the composition further comprises an agent that down-regulates expression of one or more miRNAs selected from the group consisting of miR-103*, miR-181a*, miR-20a, miR-92a, miR-17, miR30e*, miR-19b, miR-18a and miR-19a.

In another embodiment, said subject is afflicted with a disease or disorder typically treated with GCs. In another embodiment, said subject is afflicted with cancer. In another embodiment, said subject is afflicted with a cancer typically treated with GCs. In another embodiment, said cancer is selected from leukemia and lymphoma. In another embodiment, said cancer is GC-resistant.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-M: Characterization and deep sequencing analysis of glucocorticoid (GC) sensitive and resistant cells. (A) CEM-C7H2, MOLT-4, CUTLL and LOUCY T-ALL cells and (B) BJAB, SUD-136 and DAUDI Burkitt's lymphoma were untreated or treated with Dexamethasone (Dex) for 72 hours (hrs). Percentage of apoptotic cells was determined by flow cytometry of propidium iodide (PI) stained cells. (C) Relative quantification (RQ) of FKBP5 and SGK1 measured by syber green enzyme. (D) CEM-C7H2 and MOLT4 cells were untreated or treated with Dex and then subjected to western blot analysis with anti-GRα, c-Myc and BIM antibodies. (E) A fold change deep sequencing output of miRNAs that were significantly affected by 24 hrs Dex treatment of CEM-C7H2 cells, as compared to MOLT-4 cells. (F) Total numbers of miRNAs in untreated and Dex treated, CEM-C7H2 and MOLT-4 cells. (G) BJAB (H) MOLT-4 cells transfected with various overexpression or sponge plasmids were untreated or treated with Dex for 72 hrs and percentage of apoptotic cells were analyzed by flow cytometry of PI stained cells. (I-J) Fold change of GC-regulated miRNAs in CEM-C7H2 and MOLT-4 cells in a same scale range (K) The most significantly regulated miRNAs in MOLT-4 upon Dex treatment. (L) miRNA overexpression and sponge plasmids. Overexpression of miRNAs with GFP as a marker is driven by a U6 promoter whereas puromycin resistance marker (pLKO) is driven by H1 promoter. Sponge plasmids were constructed with six repeats of an imperfect miRNA antisense in the 3′UTR of the GFP gene. (M) The PANK3 gene is depicted with exons as length rectangles, introns as lines and UTRs as width rectangles. miR-103 is encoded in the intron between the fourth and fifth exons and marked red. All results are statistically significant (p≦0.001), except some of those that are presented in the table. C=Untreated, D=Dex-treated.

FIGS. 2A-S. miR-103 conferred GC induced apoptosis (GCIA) and is regulated by binding of glucocorticoid receptor (GR) to PANK3 enhancer. miR-103 overexpressing CEM-C7H2 (A) DAUDI (B), LOUCY (C) MOLT-4 (D), CUTLL (E), BJAB (F) and SUD136 (G) were untreated or treated with Dex and percentage of apoptotic cells was analyzed by flow cytometry of PI stained cells. CEM-C7H2 (A) and DAUDI (B) were transfected with sponge of miR-103 and treated with Dex for 72 hrs and % apoptotic cells were analyzed by PI staining. (H) RQ of miR-103 and PANK3 in cells treated with Dex and/or the GR inhibitor RU486. Relative quantification of (I) miR-103 and (J) PANK3 mRNA in untreated and Dex treated cells. (K) Chromatin of untreated and Dex treated CEM-C7H2 cells was immunoprecipitated with anti-GRα, controls anti-H3K4me1 (positive) or anti-IgG (negative) antibodies. DNA was then subjected to qRT-PCR with primers of the PANK3-GRE, GR-GRE and MYOD1 promoters. Percent enrichment relative to input was measured. (L) RQ of c-Myc in Dex treated cells. (M) RQ of c-Myc mRNA in control and miR-103 overexpressing BJAB cells treated with Dex. (N-O) Fold change of miR-17˜92a expression following Dex treatment of CEM-C7H2 (N) and MOLT-4 (O) cells. (P-S) miR-103 overexpressing BJAB cells were transfected with either miR-17 (P), miR-19a (Q), miR-19b (R) or miR-92a (S). The cells were treated with Dex for 72 hrs and percent of apoptotic cells was determined by PI staining.

FIGS. 3A-H: miR-103 downregulates c-Myc and miR-17˜92a expression via inhibiting DVL1 and c-Myb. (A) BJAB, CEM-C7H2, CUTLL, SUD136 and MOLT-4 cells were transfected with miR-103 overexpression or sponge plasmids. The cells were untreated or treated with Dex and subjected to western blot analysis using anti-c-Myc antibody. (B) Percent of PI stained BJAB cells transfected with Sh-c-Myc and treated with Dex. (C) Binding sites for miR-103 in the 3′UTR of DVL1 and MYB. (D) Western blot analysis of Dex treated CEM-C7H2 and MOLT-4 cells using anti-DVL1, β-Catenin and c-Myb antibodies. (E) BJAB, SUD136, MOLT-4 and CEM-C7H2 cells were transfected with miR-103 overexpression or sponge plasmids. The cells were treated with Dex and subjected to western blot analysis using anti-DVL1, β-Catenin and c-Myb antibodies. (F) Decreased RQ of miR-17˜92a members measured in miR-103 overexpressing BJAB cells as compared with control cells. (GH) miR-103 overexpressing BJAB cells were transfected with miR-20a (G) or miR-18a (H). The cells were then treated with Dex for 72 hrs and percent apoptotic cells were determined by PI staining. C=Untreated, D=Dex-treated.

FIGS. 4A-G: miR-103 upregulates GR via miR-18a downregulation. (A) A binding site for miR-18a in the 3′UTR of GR. (B) miR-103 overexpressing BJAB and CEM-C7H2 cells were treated with Dex and/or RU486 for 72 hrs. Percent apoptosis was determined by PI staining and flow cytometry (C-D) Relative quantification of miR-18a and GR in untreated or Dex treated cells. (E) Western blot analysis of GRα in miR-103 overexpressing cells treated with Dex. (F) Western blot analysis of GRα in miR-103 sponged CEM-C7H2 cells treated with Dex. (G) Western blot analysis of GRα in miR-103 overexpressing cells transfected with miR-18a and treated with Dex. C=Untreated, D=Dex-treated.

FIGS. 5A-G: miR-103 upregulates BIM via miR-20a downregulation. (A) Two conserved binding sites for miR-20a in the 3′UTR of BIM mRNA. (B-C) Relative quantification of miR-20a (B) and BIM (C) mRNA in Dex treated cells. (D) Western blot analysis of BIMEL in miR-103 overexpressing or sponge cells treated with Dex. (E) miR-103 overexpressing BJAB cells were transfected with Sh-BIM and treated with Dex for 72 hrs. Percent of apoptotic cells was determined by PI staining. (F) Western blot analysis of BIMEL in miR-103 overexpressing BJAB cells transfected with Sh-BIM and treated with Dex. (G) Western blot analysis of BIMEL in miR-103 overexpressing BJAB cells which were transfected with miR-20a and treated with Dex. C=Untreated, D=Dex-treated.

FIGS. 6A-E: miR-103 inhibits cellular proliferation. (A) Relative Quantification of miR-103 in BM cells of 12 ALL patients and 4 healthy donors. (B) CEM-C7H2, BJAB and miR-103 overexpressing BJAB cells were treated with Dex (24 hrs), labeled with BRDU (1 hr), fixed and analyzed by flow cytometry. (C) miR-103 overexpressing BJAB, SUD-136, DAUDI and MOLT-4 cells were treated with Dex for 72 hrs. Cellular proliferation was measured by BRDU staining. (D) miR-103 overexpressing BJAB cells were treated with Dex and/or RU486 for 72 hrs. Cell viability was determined by flow cytometry. (E) miR-103 overexpression or sponged cells were treated with Dex and then subjected to western blot analysis using anti Cyclin E1 and CDK2 antibodies. C=Untreated, D=Dex-treated.

FIG. 7: A model of miR-103 network in GC induced apoptosis (GCIA). GC upregulates miR-103 expression by direct binding of activated GR to a GRE sequence in the promoter of its host gene PANK3. miR-103 inhibits CDK2 and Cyclin E1 translation thus reducing cellular proliferation. In addition, miR-103 inhibits c-Myb and/or DVL1 by binding to their 3′UTR. c-Myb downregulation results with c-Myc transcription inhibition, whereas DVL1 downregulation results with β-Catenin degradation and transcription inhibition of c-Myc. c-Myc ablation is followed by downregulation of miR-18a and miR-20a. As miR-18a inhibits GR translation, expression of GR is upregulated. GR accumulation accelerates the response to GC and further elevates miR-103 in a positive feedback pathway. miR-20a inhibits BIM translation and, therefore, miR-103-induced miR-20a downregulation is followed by BIM upregulation. Pro-apoptotic BIM activates the mitochondrial apoptotic pathway by it inhibitory interaction with Bcl-2 proteins, which is followed by BAK and BAK oligomerization, release of cytochrom C and SMAC/Diablo, thus leading to GCIA.

FIG. 8: miR-103 level in peripheral blood lymphoblasts (PBLs) treated with Prednisone (PRED) or Dexamethasone (Dex) compared to untreated PBLs. Blood samples (depicted as ND-1, ND-2, R-B-ALL, B-ALL-1-4, T-ALL and CLL) were treated with Dex for 24 h. Blood samples depicted as “B-ALL-5” and “B-ALL-6” were taken at day 0 (before treatment) and day 1 following Prednisone treatment.

FIGS. 9A-G: Relative quantification of GR mRNA (NR3C1) (A) or BIM mRNA (BCL2L11) (B) in miR-103 overexpressing cells (both pLKO and SinGFP) untreated or treated with Dex. (C-E) RQ of miR-18a (C), GR (D) and BIM (E) in miR-103 overexpressing BJAB cells which were transfected with c-Myc overexpression and treated with Dex. (F-G) Relative Quantification of cycline E1 (F) and CDK2 (G) in miR-103 overexpressing BJAB cells treated with Dex.

FIG. 10: miR-103 expression in various human organs.

### DETAILED DESCRIPTION OF THE INVENTION

The present invention provides methods of determining miRNAs levels for predicting responsiveness of a subject to glucocorticoids (GCs) treatment. The present invention further provides pharmaceutical compositions and methods for GCs therapy, including but not limited to, improving a therapeutic response to GC treatment in a subject in need thereof.

Glucocorticoids are steroid hormones characterized by their ability to bind to the cortisol receptor. Glucocorticoids are well known to those of skill in the art and any glucocorticoid known to those of skill in the art with these characteristics can be used in the compositions and methods of the present invention. The clinical use of glucocorticoids is described, for example, in detail in the Physicians' Desk Reference, 56^{th} Ed. (2002) Publisher Edward R. Barnhart, N.J.

Non-limiting examples of GCs include, but are not limited to, betamethasone, budesonide, cortisone, cortisone acetate, dexamethasone, hydrocortisone, methylprednisolone, prednisolone, prednisone, triamcinolone, methylprednisolone, triamcinolone, beclomethasone, fludrocortisone acetate, deoxycorticosterone acetate (DOCA) and aldosterone.

The term “glucocorticoid” or “glucocorticosteroid” is intended to denote a therapeutically, prophylactically and/or diagnostically active glucocorticoid or a glucocorticoid that has physiologic effect. The term is intended to include the glucocorticoid in any suitable form such as e.g. a pharmaceutically acceptable salt, complex, solvate, ester, active metabolites or prodrug thereof of in any physical form such as, e.g., in the form of crystals, amorphous or a polymorphous form or, if relevant, in any stereoisomer form including any enantiomeric or racemic form, or a combination of any of the above. The glucocorticoid may be a synthetic glucocorticoid.

As used herein the phrases “GC treatment” “GC therapy” and other grammatical derivatives thereof interchangeably used herein, refers to administration of one or more types of GCs to a subject in need thereof. It should be noted that administration of GCs may comprise a single or multiple dosages, as well as a continuous administration, depending on the pathology to be treated and the subject receiving the treatment.

The term “patient” or “subject” as used herein refers to a mammal, preferably a human being (male or female) at any age. In some embodiments, the subject is diagnosed with a pathology (disease, disorder or condition) which requires GCs treatment such as cancer, as described above. One skilled in the art understands that the methods of the invention can be applied to a variety of diseases and disorders in which GCs are typically used to induce cell apoptosis such as allergic disorders, autoimmune diseases, hematopoietic cancers, osteosarcoma, lung cancer, to name a few.

According to some embodiments of the invention, the subject is a healthy subject (e.g., not diagnosed with any disease which require GCs treatment). It should be noted that determining the responsiveness of a healthy subject to GCs treatment can be performed as part of a genetic testing of the healthy subject, which can be recorded in the subject's medical file for future use (e.g., in case the subject will be diagnosed with a disease requiring GCs treatment).

miRNAs

As used interchangeably herein, the terms “microRNA”, “miRNA” and “miR-” refer to non-coding single-stranded RNA molecules having a nucleotide sequence that is complementary in whole or in part to one or more messenger RNA (mRNA) molecules.

miRNAs arise from intergenic or intragenic (both exonic and intronic) genomic regions that are transcribed as long primary transcripts (pri-microRNA) and undergo a number of processing steps to produce the final short mature molecule (Massimo et al., Current Op. in Cell Biol. 2009: 21; 1-10). As known to one skilled in the in the art, a gene coding for a miRNA may be transcribed leading to production of a miRNA precursor known as the pri-miRNA. The pri-miRNA is typically a part of a polycistronic RNA comprising multiple pri-miRNAs. The pri-miRNA may form a hairpin with a stem and loop. The stem may comprise mismatched bases. The hairpin structure of the pri-miRNA may be recognized by Drosha, which is an RNase III endonuclease. Drosha may recognize terminal loops in the pri-miRNA and cleave approximately two helical turns into the stem to produce a 30-200 nt precursor known as the pre-miRNA. Drosha may cleave the pri-miRNA with a staggered cut typical of RNase III endonucleases yielding a pre-miRNA stem loop with a 5′ phosphate and ^{˜}2 nucleotide 3′ overhang. Approximately one helical turn of stem (^{˜}10 nucleotides) extending beyond the Drosha cleavage site may be essential for efficient processing. The pre-miRNA may then be actively transported from the nucleus to the cytoplasm by Ran-GTP and the export receptor Ex-portin-5.

In some embodiments, the miRNAs or precursor miRNAs utilized in the methods, kits and compositions of the present invention are human miRNAs, such as listed in Tables 1-2 below.

TABLE 1
Exemplary miRNAs

| miRNA | Accession number | SEQ ID NO: | Sequence |
|---|---|---|---|
| hsa-miR-103 | MIMAT0000101 | 1 | AGCAGCAUUGUACAGGGCUAUGA |
| hsa-miR-30e | MIMAT0000692 | 2 | UGUAAACAUCCUUGACUGGAAG |
| hsa-miR-30d | MIMAT0000245 | 3 | UGUAAACAUCCCCGACUGGAAG |
| hsa-miR-15b | MIMAT0000417 | 4 | UAGCAGCACAUCAUGGUUUACA |
| hsa-miR-16 | MIMAT0000069 | 5 | UAGCAGCACGUAAAUAUUGGCG |
| hsa-miR-181a* | MIMAT0000270 | 6 | ACCAUCGACCGUUGAUUGUACC |
| hsa-miR-15b* | MIMAT0004586 | 7 | CGAAUCAUUAUUUGCUGCUCUA |
| hsa-miR-21 | MIMAT0000076 | 8 | UAGCUUAUCAGACUGAUGUUGA |
| hsa-miR-181a | MIMAT0000256 | 9 | AACAUUCAACGCUGUCGGUGAGU |
| hsa-let-7f | MIMAT0000067 | 10 | UGAGGUAGUAGAUUGUAUAGUU |
| hsa-miR-186 | MIMAT0000456 | 11 | CAAAGAAUUCUCCUUUUGGGCU |
| hsa-miR-223 | MIMAT0004570 | 12 | CGUGUAUUUGACAAGCUGAGUU |
| hsa-miR-103* | MIMAT0007402 | 13 | UCAUAGCCCUGUACAAUGCUGCU |
| hsa-miR-20a | MIMAT0000075 | 14 | UAAAGUGCUUAUAGUGCAGGUAG |
| hsa-miR-92a | MIMAT0004507 | 15 | AGGUUGGGAUCGGUUGCAAUGCU |
| hsa-miR-17 | MIMAT0000070 | 16 | CAAAGUGCUUACAGUGCAGGUAG |
| hsa-miR-30e* | MIMAT0000693 | 17 | CUUUCAGUCGGAUGUUUACAGC |
| hsa-miR-19b | MIMAT0004491 | 18 | AGUUUUGCAGGUUUGCAUCCAGC |
| hsa-miR-18a | MIMAT0000072 | 19 | UAAGGUGCAUCUAGUGCAGAUAG |
| hsa-miR-19a | MIMAT0004490 | 20 | AGUUUUGCAUAGUUGCACUACA |

TABLE 2
Exemplary pre-miRNA

| | Accession | SEQ | |
|---|---|---|---|
| Pre-miRNA | number | ID NO: | Sequence |
| Pre-miR-103 | MI0000109 | 21 | UACUGCCCUCGGCUUCUUUACAGUGCUGCCUUGUUGC |
| | | | AUAUGGAUCAAGCAGCAUUGUACAGGGCUAUGAAGGC |
| | | | AUUG |
| Pre-miR-30e | MI0000749 | 22 | GGGCAGUCUUUGCUACUGUAAACAUCCUUGACUGGAA |
| | | | GCUGUAAGGUGUUCAGAGGAGCUUUCAGUCGGAUGUU |
| | | | UACAGCGGCAGGCUGCCA |
| Pre-miR-30d | MI0000255 | 23 | GUUGUUGUAAACAUCCCCGACUGGAAGCUGUAAGACA |
| | | | CAGCUAAGCUUUCAGUCAGAUGUUUGCUGCUAC |
| Pre-miR-15b | MI0000438 | 24 | UUGAGGCCUUAAAGUACUGUAGCAGCACAUCAUGGUU |
| | | | UACAUGCUACAGUCAAGAUGCGAAUCAUUAUUUGCUG |
| | | | CUCUAGAAAUUUAAGGAAAUUCAU |
| Pre-miR-16 | MI0000070 | 25 | GUCAGCAGUGCCUUAGCAGCACGUAAAUAUUGGCGUU |
| | | | AAGAUUCUAAAAUUAUCUCCAGUAUUAACUGUGCUGC |
| | | | UGAAGUAAGGUUGAC |
| Pre-miR-181a* | MI0000269 | 26 | AGAAGGGCUAUCAGGCCAGCCUUCAGAGGACUCCAAG |
| | | | GAACAUUCAACGCUGUCGGUGAGUUUGGGAUUUGAAA |
| | | | AAACCACUGACCGUUGACUGUACCUUGGGGUCCUUA |
| Pre-miR-15b* | MI0000438 | 27 | UUGAGGCCUUAAAGUACUGUAGCAGCACAUCAUGGUU |
| | | | UACAUGCUACAGUCAAGAUGCGAAUCAUUAUUUGCUG |
| | | | CUCUAGAAAUUUAAGGAAAUUCAU |
| Pre-miR-21 | MI0000077 | 28 | UGUCGGGUAGCUUAUCAGACUGAUGUUGACUGUUGAA |
| | | | UCUCAUGGCAACACCAGUCGAUGGGCUGUCUGACA |
| Pre-miR-181a | MI0000269 | 29 | AGAAGGGCUAUCAGGCCAGCCUUCAGAGGACUCCAAG |
| | | | GAACAUUCAACGCUGUCGGUGAGUUUGGGAUUUGAAA |
| | | | AAACCACUGACCGUUGACUGUACCUUGGGGUCCUUA |
| Pre-let-7f | MIMAT0000067 | 30 | UCAGAGUGAGGUAGUAGAUUGUAUAGUUGUGGGGUAG |
| | | | UGAUUUUACCCUGUUCAGGAGAUAACUAUACAAUCUA |
| | | | UUGCCUUCCCUGA |
| Pre-miR-186 | MI0000483 | 31 | UGCUUGUAACUUUCCAAAGAAUUCUCCUUUUGGGCUU |
| | | | UCUGGUUUUAUUUUAAGCCCAAAGGUGAAUUUUUUGG |
| | | | GAAGUUUGAGCU |
| Pre-miR-223 | MI0000300 | 32 | CCUGGCCUCCUGCAGUGCCACGCUCCGUGUAUUUGAC |
| | | | AAGCUGAGUUGGACACUCCAUGUGGUAGAGUGUCAGU |
| | | | UUGUCAAAUACCCCAAGUGCGGCACAUGCUUACCAG |
| Pre-miR-103* | MI0007261 | 33 | UCAUAGCCCUGUACAAUGCUGCUUGAUCCAUAUGCAA |
| | | | CAAGGCAGCACUGUAAAGAAGCCGA |
| Pre-miR-20a | MI0000076 | 34 | GUAGCACUAAAGUGCUUAUAGUGCAGGUAGUGUUUAG |
| | | | UUAUCUACUGCAUUAUGAGCACUUAAAGUACUGC |
| Pre-miR-92a | MI0000093 | 35 | CUUUCUACACAGGUUGGGAUCGGUUGCAAUGCUGUGU |
| | | | UUCUGUAUGGUAUUGCACUUGUCCCGGCCUGUUGAGU |
| | | | UUGG |
| Pre-miR-17 | MI0000071 | 36 | GUCAGAAUAAUGUCAAAGUGCUUACAGUGCAGGUAGU |
| | | | GAUAUGUGCAUCUACUGCAGUGAAGGCACUUGUAGCA |
| | | | UUAUGGUGAC |
| Pre-miR-30e* | MI0000749 | 37 | GGGCAGUCUUUGCUACUGUAAACAUCCUUGACUGGAA |
| | | | GCUGUAAGGUGUUCAGAGGAGCUUUCAGUCGGAUGUU |
| | | | UACAGCGGCAGGCUGCCA |
| Pre-miR-19b | MI0000074 | 38 | CACUGUUCUAUGGUUAGUUUUGCAGGUUUGCAUCCAG |
| | | | CUGUGUGAUAUUCUGCUGUGCAAAUCCAUGCAAAACU |
| | | | GACUGUGGUAGUG |
| Pre-miR-18a | MI0000072 | 39 | UGUUCUAAGGUGCAUCUAGUGCAGAUAGUGAAGUAGA |
| | | | UUAGCAUCUACUGCCCUAAGUGCUCCUUCUGGCA |
| Pre-miR-19a | MI0000073 | 40 | GCAGUCCUCUGUUAGUUUUGCAUAGUUGCACUACAAG |
| | | | AAGAAUGUAGUUGUGCAAAUCUAUGCAAAACUGAUGG |
| | | | UGGCCUGC |

In some embodiments, the methods, kits and compositions of the inventions comprise the step of determining or modulating the expression level of at least one miRNAs selected from the group consisting of miR-103, miR-30e, miR-30d, miR-181a*, miR-15b* and miR-21.

In additional embodiments, the methods, kits and compositions of the inventions comprise the step of determining or modulating the expression level of a plurality of miRNAs selected from Tables 1-2.

In one embodiment, the methods, kits and compositions described herein comprise the step of determining or modulating the expression level of miR-103. In one embodiment, the methods, kits and compositions described herein comprise the step of determining or modulating the expression level of miR-103 and at least one miR selected from miR-30e and miR-30d.

In one embodiment, the plurality of miRNAs of the invention comprises miR-103 and at least one miRNA selected from the group consisting of miR-30e, miR-30d, miR-15b and miR-16. In one embodiment, the plurality of miRNAs of the invention comprises miR-103 and at least one miRNA selected from the group consisting of miR-103*, miR-30e, miR-30d, miR-15b, miR-16, miR-20a, miR-15b*, miR-181a, miR-181a*, miR-92a, miR-17, miR-19b, miR-18a, miR-19a, miR-213, let-7f, miR-21, miR-30e*, miR-186. In one embodiment, the plurality of miRNAs of the invention comprises miR-103 and miR-103*. In one embodiment, the plurality of miRNAs of the invention comprises miR-103, miR-103* and at least one miRNA selected from the group consisting of miR-103*, miR-30e, miR-30d, miR-15b, miR-16, miR-20a, miR-15b*, miR-181a, miR-181a*, miR-92a, miR-17, miR-19b, miR-18a, miR-19a, miR-213, let-7f, miR-21, miR-30e* and miR-186.

In one embodiment, the methods, kits and compositions described herein comprise the step of determining or modulating the expression level of miR-30e. In one embodiment, the methods, kits and compositions described herein comprise the step of determining or modulating the expression level of miR-30e and at least one miR selected from miR-103 and miR-30d. In one embodiment, the plurality of miRNAs of the invention comprises miR-30e and at least one miR selected from the group consisting of miR-103, miR-103*, miR-30d, miR-15b and miR-16. In one embodiment, the plurality of miRNAs of the invention comprises miR-103 and at least one miRNA selected from the group consisting of miR-103, miR-103*, miR-30d, miR-15b, miR-16, miR-20a, miR-15b*, miR-181a, miR-181a*, miR-92a, miR-17, miR-19b, miR-18a, miR-19a, miR-213, let-7f, miR-21, miR-30e*, miR-186.

In one embodiment, the methods, kits and compositions described herein comprise the step of determining or modulating the expression level of miR-30d. In one embodiment, the methods, kits and compositions described herein comprise the step of determining or modulating the expression level of miR-30d and at least one miR selected from of miR-103 and miR-30e. In one embodiment, the plurality of miRNAs of the invention comprises miR-30e and at least one miR selected from the group consisting of miR-103, miR-103*, miR-30e, miR-15b and miR-16. In one embodiment, the plurality of miRNAs of the invention comprises miR-103 and at least one miRNA selected from the group consisting of miR-103, miR-103*, miR-15b, miR-16, miR-20a, miR-15b*, miR-181a, miR-181a*, miR-92a, miR-17, miR-19b, miR-18a, miR-19a, miR-213, let-7f, miR-21, miR-30e*, miR-186.

In one embodiment, the plurality of miRNA of the invention comprises at least one miRNAs selected from miR-103, miR-30e, miR-30d, miR-181a*, miR-15b* and miR-21. In one embodiment, the plurality of miRNAs of the invention comprises at least two miRNAs selected from miR-103, miR-30e, miR-30d, miR-181a*, miR-15b* and miR-21. In one embodiment, the plurality of miRNAs of the invention comprises at least three miRNAs selected from miR-103, miR-30e, miR-30d, miR-181a*, miR-15b* and miR-21. In one embodiment, the plurality of miRNAs of the invention comprises at least four miRNAs selected from miR-103, miR-30e, miR-30d, miR-181a*, miR-15b* and miR-21. In one embodiment, the plurality of miRNAs of the invention comprises at least five miRNAs selected from miR-103, miR-30e, miR-30d, miR-181a*, miR-15b* and miR-21. In one embodiment, the plurality of miRNAs of the invention comprises miR-103, miR-30e and miR-30d. In one embodiment, the plurality of miRNAs of the invention comprises miR-103, miR-30e, miR-30d, miR-181a*, miR-15b* and miR-21.

In one embodiment, the plurality of miRNAs of the invention further comprises miR-15b. In one embodiment, the plurality of miRNAs of the invention further comprises miR-16. In one embodiment, the plurality of miRNAs of the invention further comprises miR-103*. In one embodiment, the plurality of miRNAs of the invention further comprises miR-20a. In one embodiment, the plurality of miRNAs of the invention further comprises miR-15b*. In one embodiment, the plurality of miRNAs of the invention further comprises miR-181a. In one embodiment, the plurality of miRNAs of the invention further comprises miR-92a. In one embodiment, the plurality of miRNAs of the invention further comprises miR-17. In one embodiment, the plurality of miRNAs of the invention further comprises miR-19b. In one embodiment, the plurality of miRNAs of the invention further comprises miR-18a. In one embodiment, the plurality of miRNAs of the invention further comprises miR-19a. In one embodiment, the plurality of miRNAs of the invention further comprises miR-223. In one embodiment, the plurality of miRNAs of the invention further comprises miR-30e*. In one embodiment, the plurality of miRNAs of the invention further comprises miR-186. In one embodiment, the plurality of miRNAs of the invention further comprises let-7f.

In another embodiment, the miRNAs of the invention have the nucleic acid sequences as known in the art or sequences of at least 80%, at least 85%, at least 90% or at least 95% identical thereto. Each possibility is a separate embodiment of the invention.

One skilled in the art will appreciate that the methods of the invention may be employed on one or more pre-miRNAs of the invention, or sequences having at least 80%, at least 85%, at least 90% or at least 95% identity thereto. Each possibility is a separate embodiment of the invention. In one embodiment of the methods and kits of the invention, the nucleic acid sequences of the miRNAs are selected from pre-miR103, pre-miR30e and pre-miR30d. In some embodiments of the methods and kits of the invention, the plurality of miRNAs further comprises pre-miR15b and/or pre-miR16.

### Responsiveness to Glucocorticoids Therapy

According to some embodiments, the present invention provides methods and kits for predicting responsiveness of a subject to GC treatment. As used herein the phrase “predicting responsiveness of a subject to GCs treatment” refers to determining the likelihood that the subject will respond to GCs treatment, e.g., the success or failure of GCs treatment.

The term “response” to GCs treatment refers to an improvement in at least one relevant clinical parameter as compared to an untreated subject diagnosed with the same pathology (e.g., the same type, stage, degree and/or classification of the pathology), or as compared to the clinical parameters of the same subject prior to GCs treatment.

In some embodiments, a “low probability of response to GCs treatment” is a probability significantly lower than about 50%, e.g., a probability lower than about 40%, e.g., a probability lower than about 30%, e.g., a probability lower than about 20%, e.g., a probability lower than about 10% or 5%, and a “high probability of response to GCs treatment” is a probability significantly higher than about 50%, e.g., a probability higher than about 60%, e.g., a probability higher than about 70%, e.g., a probability higher than about 80%, e.g., a probability higher than about 85%, e.g., a probability higher than about 90%, e.g., a probability higher than about 95%, e.g., a probability higher than about 99%.

As exemplified herein below, deep sequencing analysis of small RNA revealed that GCs regulate miRNA expression. Surprisingly, GC-induced miRNA alteration occurs only in GC-sensitive but not in GC-resistant cell lines, even though they both express an intact GR. One skilled in the art will recognize that identification of a GC resistant patient will enable a physician to choose alternative therapies, thus optimizing the efficacy of the treatment while avoiding adverse effects mediated by an unnecessary GC treatment.

In some embodiments, there is provided a method of predicting the responsiveness of a subject to GC treatment, the method comprises determining the expression level of one or more miRNAs selected from the group consisting of miR-103, miR-30e, miR-30d, miR-181a*, miR-15b* and miR-21, wherein modulated expression levels of one or more miRNAs compared to control indicates that the subject will be responsive to GC treatment.

In another embodiment, the method comprises determining the expression level of miR-103, wherein increased expression levels of miR-103 compared to control indicates that the subject will be responsive to GC treatment.

As used herein, throughout the text, the term “modulated” or “modulation” means either an increase or a decrease in the expression of miRNA. The modulation may be determined by comparing the amount or concentration of one or more miRNAs of the invention in a sample to an expression level of a control sample or to a predetermined value of expression level.

In some embodiments, the one or more miRNAs are selected from the group consisting of: miR-103, miR-30e, miR-30d, miR-15b* and miR-21, miR-15b, miR-16, miR-181a, let-7f, miR-186, miR-223, and the modulation is an increase in expression, e.g., a statistically significant increase in expression levels.

In another embodiment, the one or more miRNAs are selected from the group consisting of: miR-181a*, miR-103*, miR-20a, miR-92a, miR-17, miR30e*, miR-19b, miR-18a and miR-19a, and the modulation is a decrease in expression, e.g., a statistically significant decrease in expression levels.

As used herein, the term “determining” and grammatical derivatives thereof, such as, but not limited to “determine” or “determined” can include measuring the expression level, for example, the amount or concentration of one or more miRNAs of the invention. Determination of the level of a miR can include comparing the level of one or more miRNAs in a sample with the level of the marker in a control sample or with the level of the marker obtained from the same patient but at a different time point.

According to another embodiment, the control value (of each miRNA) correlates to the expression level of said miRNA in a sample sensitive to GC treatment (e.g., obtained from the subject being diagnosed or from a healthy subject). The control value may be a miRNA-specific predetermined threshold.

As used herein the phrase “predetermined threshold” or “reference expression data” refers to the expression level of a specific miRNA in a cell of a subject who's responsiveness to GCs is already known (e.g., a reference responder or non-responder subject). Such as an expression level can be known from the literature, from a database or from biological samples comprising miRNA obtained from a reference responder or non-responder subject.

Typically, the comparison to the control values is performed in a sample (e.g., tissue, fluid or excretion) specific manner. In one embodiment, the methods further comprise comparing miRNA expression levels in the sample to miRNA expression levels in a normal reference sample. In one embodiment, the reference sample is a biological sample from the subject being diagnosed or treated. In one embodiment, the reference sample may be a biological sample from a second subject.

As used herein, the term “sample” generally refers to a biological material, that comprises nucleic acids (e.g., miRNA), being tested for and/or suspected of containing cancer cells. The biological material may be derived from any biological source. Examples of biological materials include, but are not limited to, a cell lysate, a cell culture, a cell line, a tissue, a biological fluid, a blood sample, a serum sample, a plasma sample, a urine sample, a bone marrow sample, a skin sample, fresh frozen tissue sample, a paraffin embedded tissue sample or an extract or processed sample produced from any of a peripheral blood sample, a serum or a plasma fraction of a blood sample. The test sample may be used directly as obtained from the biological source or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc.

In some embodiments, peripheral blood lymphocytes are separated from peripheral blood by methods well known to a person skilled in the art. In some embodiments, separation of PBLs is achieved by using commercial kits utilizing a density gradient such as ficoll-paque PLUS (GE Healthcare).

In one embodiment, the sample is a blood sample. In one embodiment, the sample is a serum sample. In one embodiment, the sample is a plasma sample.

One skilled in the art will appreciate that blood cells comprise heterogeneous populations having different characteristics. Therefore, in some embodiments, in order to predict the responsiveness of cells to GCs treatment there is a need to evaluate the expression of miRs specifically in each population of cells. For the purpose of examining specific populations of cells, various assays and techniques well known in the art (e.g., flow cytometry, FACS) may be used. In some embodiments, the methods and kits of the invention comprise determining the expression levels of miRs in a single cell resolution.

In some embodiments, the methods and kits of the invention comprise analyzing specific cell populations (e.g. B-cells, T-cells) for the expression levels of the miRs disclosed herein. A specific population of hematopoietic cells can be differentiated by virtue of surface membrane proteins which are characteristic of the cells. As a non-limiting example, particularly for hematopoietic cells, Cluster of differentiation (CD)34 is a marker for immature hematopoietic cells. Markers for dedicated cells include CD 10, CD19, CD20, and surface immunoglobulin (sIg) for B cells, CD 15 for granulocytes, CD 16 and CD33 for myeloid cells, CD 14 for monocytes, CD41 for megakaryocytes, CD38 for lineage dedicated cells, CD3, CD4, CD7, CD8 and T cell receptor (TCR) for T cells, Thy-1 for progenitor cells, glycophorin for erythroid progenitors and CD71 for activated T cells.

One of skill in the art would readily recognize that the measurement of the different markers can be accomplished using various types of assays well-known in the art. In some embodiments, the markers are detected using a specific binding agent, such as an antibody. In another embodiment, the assay is an immunoassay such as an enzyme-linked immunosorbent assay (ELISA) or sandwich-type ELISA. In another embodiment, the assay is flow cytometry.

According to some embodiments of the invention, the RNA molecules (particularly miRNAs) are extracted from the cell of the subject.

Methods of extracting RNA molecules from cells of a subject are well known in the art. Once obtained, the RNA molecules can be characterized for the expression and/or activity level of various RNA molecules using methods known in the arts.

Non-limiting examples of methods of detecting RNA molecules (particularly miRNAs) in a sample include Northern blot analysis, single-molecule detection flow cytometric methods, multiplex and/or singleplex RT-PCR, RNA in situ hybridization (using e.g., DNA or RNA probes to hybridize RNA molecules present in the cells or tissue sections), in situ RT-PCR (e.g., as described in Nuovo G J, et al. Am J Surg Pathol. 1993, 17: 683-90; Komminoth P, et al. Pathol Res Pract. 1994, 190: 1017-25), and oligonucleotide microarray (e.g., by hybridization of polynucleotide sequences derived from a sample to oligonucleotides attached to a solid surface (e.g., a glass wafer) with addressable location, such as Affymetrix microarray (Affymetrix®, Santa Clara, Calif.)).

### Methods of Glucocorticoids (GCs) Therapy

According to some embodiments, the present invention provides methods of glucocorticoid therapy. In one embodiment, there is provided a method for treating a subject with GC, the method comprising:
i. determining the expression levels of a one or a plurality of miRNAs selected from the group consisting of miR-103, miR-30e, miR-30d, miR-181a*, miR-15b* and miR-21, in a sample obtained from a subject, wherein modulated expression levels of said one or more miRNA compared to control indicates that the subject is susceptible for GC treatment; and
ii. administering a therapeutically effective amount of GC to the susceptible subject; thereby treating said subject with GC.

According to another embodiment, the present invention provides a method of improving a therapeutic response to GC treatment in a subject in need thereof. In one embodiment, the method comprises administering to the subject a pharmaceutical composition comprising an agent selected from (i) one or a plurality of miRNAs selected from the group consisting of miR-103, miR-30e and miR-30d, miR-15b* and miR-21 (ii) an agent that up-regulates expression of said miRNAs, and (iii) a combination thereof; and a pharmaceutically acceptable carrier.

According to some embodiments, the present invention provides therapeutic compositions and methods for sensitizing subjects or cells thereof to respond to GC treatment. According to some embodiments, the present invention provides therapeutic compositions and methods for re-sensitizing subjects or cells thereof to respond to GC treatment.

As used herein, the term “sensitize” or “sensitizing” refers to a process of rendering a subject receptive to a treatment. “Re-sensitizing” refers to a process of restoring responsiveness to a treatment in a subject who was receptive to a treatment but who is no longer receptive to a treatment due to the fact that the patient or cells thereof developed a resistance to the specific therapy (e.g., GC treatment). In this context, “an effective amount” refers to an amount sufficient to sensitize or re-sensitize the patient to treatment with GCs.

According to another embodiment, the present invention provides a pharmaceutical composition useful for improving a therapeutic response to GC treatment in a subject in need thereof. According to another embodiment, the present invention provides use of pharmaceutical composition for preparation of a medicament for improving a therapeutic response to GC treatment in a subject in need thereof

According to another embodiment, the present invention provides a pharmaceutical composition comprising an agent selected from (i) one or a plurality of miRNAs selected from the group consisting of miR-103, miR-30e and miR-30d, miR-15b* and miR-21 (ii) an agent that up-regulates expression of said miRNAs, and (iii) a combination thereof; and a pharmaceutically acceptable carrier.

In another embodiment, the pharmaceutical composition further comprises one or more miRNAs selected from the group consisting of miR-15b, miR-16, miR-181a, let-7f, miR-186, miR-223. In another embodiment, the pharmaceutical composition further comprises an agent that up-regulates expression of one or more miRNAs selected from the group consisting of miR-15b, miR-16, miR-181a, let-7f, miR-186, miR-223.

In another embodiment, the pharmaceutical composition comprises an agent that acts as an miRNA inhibitor of one or more miRNAs selected from the group consisting of miR-181a*, miR-103*, miR-20a, miR-92a, miR-17, miR30e*, miR-19b, miR-18a, miR-19a.

The term “miRNA inhibitor” as used herein, refers to an agent that reduces or inhibits the expression, stability, sub-cellular localization, or activity of an miRNA. An miRNA inhibitor may function, for example, by blocking the activity of an miRNA (e.g., blocking the ability of an miRNA to function as a translational repressor of one or more miRNA targets), or by mediating miRNA degradation.

In one embodiment, the miRNA inhibitor can be selected from a variety of molecules which interfere with transcription (e.g., RNA silencing agents, Ribozyme, DNAzyme and antisense). In one embodiment, a “miRNA inhibitor” is a polynucleotide (also referred to as an oligonucleotide) having a sequence that is antisense, either complementary or partially complementary, to one or more miRNAs selected from the group consisting of miR-181a*, miR-103*, miR-20a, miR-92a, miR-17, miR30e*, miR-19b, miR-18a, miR-19a.

The terms “oligonucleotide” and “polynucleotide” are used interchangeably to refer to a polymer composed of a multiplicity of nucleotide residues (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-0-methyl ribonucleic acids, and the like.

As used herein, the term “antisense” refers to a nucleic acid sequence complementary to its respective miRNA molecule. As used herein, the terms “complementary” or “complementarity” are used in reference to polynucleotides (i.e., a sequence of nucleotides) related by the base-pairing rules. For example, the sequence “A-G-T” is complementary to the sequence “T-C-A,” and also to “T-C-U.” Complementation can be between two DNA strands, a DNA and an RNA strand, or an RNA and another RNA strand. Partial complementarity or complementation occurs when only some of the nucleic acid bases are matched according to the base pairing rules. Complete or total complementarity or complementation occurs when the bases are completely matched between the nucleic acid strands. The degree of complementarity between nucleic acid strands has significant effects on the efficiency and strength of hybridization between nucleic acid strands. This is of particular importance in amplification reactions, as well as in detection methods which depend upon binding between nucleic acids. Percent complementarity or complementation refers to the number of mismatch bases over the total bases in one strand of the nucleic acid. Thus, a 50% complementation means that half of the bases were mismatched and half were matched. Two strands of nucleic acid can be complementary even though the two strands differ in the number of bases. In this situation, the complementation occurs between the portion of the longer strand corresponding to the bases on that strand that pairs with the bases on the shorter strand.

In some embodiments of the invention, the miRs of the invention or one or more agents that modulate (up- or down-regulates) miRNA expression are provided within a delivery vehicle. Non-limiting examples of delivery vehicle suitable for miRNA expression modulation include a viral vector, microspheres, liposomes, colloidal gold particles, lipopolysaccharides, polypeptides, polysaccharides, or pegylation of viral vehicles.

In one embodiment, the patient is insensitive to GC treatment. As used herein the term “insensitive” refers to a patient or a patient's cell which is, to at least some degree, refractory to treatment with a particular therapy. The term “insensitive” therefore is used to describe a patient or a cells thereof otherwise referred to as resistant, for example, GC-resistant. One skilled in the art will recognize that the term “insensitive” also includes subjects who display diminished responsiveness to GCs therapy when compared to sensitive patients and cells thereof. As known in the art, the dose regimen of subject undergoing GCs therapy is typically increased over time due to GCs insensitivity.

As used herein, the terms “subject” or “individual” or “animal” or “patient” or “mammal,” refers to any subject, particularly a mammalian subject, for whom diagnosis, prognosis, or therapy is desired, for example, a human.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

In one embodiment of the invention, the patient is a cancer patient. In one embodiment of the invention, the patient is a primary diagnosed patient or alternatively a relapse or refractory patient. As used herein, “cancer patient” refers to a subject who has been diagnosed with any type of cancer or has been given a probable diagnosis of cancer. As used herein, “cancer” describes the physiological condition that is typically characterized by unregulated cell growth. The cancer may be benign and/or malignant. As used herein, “relapsed or refractory” refers to a type of cancer that is resistant to treatment with an agent, or responds to treatment with an agent but comes back without being resistant to that agent, or responds to treatment with an agent but comes back resistant to that agent.

In one embodiment of the invention, the patient is a blood cancer patient. In one embodiment of the invention, the patient is a leukemia patient. In one embodiment of the invention, the patient is a lymphoma patient.

As used interchangeably herein “blood cancer”, “hematological malignancy”, “hematological cancer”, “hematopoietic malignancy” and “hematopoietic cancer” refer to a hematological malignancy involving abnormal hyperproliferation or malignant growth and/or metastasis of a blood cell. Blood cancers include, without limitation, leukemia, lymphoma, and myeloma and specific disease types thereof. In some embodiments, the present invention is concerned with hematopoietic cancers that are typically treated with GCs. In some embodiment, the present invention is concerned with hematopoietic cancers of the lymphoid lineage (e.g., T-cells, B-cells, NK-cells). Hematopoietic cancers of the lymphoid lineage Acute Lymphoblastic Leukemia (ALL), Chronic lymphocytic leukemia (CLL), and multiple myeloma Hodgkin's lymphoma, non-Hodgkin's lymphoma (small-cell type, large-cell type, and mixed-cell type), Burkitt's lymphoma and T cell lymphoma.

“Acute” in the context of blood cancers, refers to the relatively short time course in which these cancers can become extremely serious and even lead to the death of a patient (e.g., they can be fatal in as little as a few weeks if left untreated) and differentiates them from “chronic” blood cancers, which may not have extremely debilitating effects on or lead to the death of a patient for many years. “Acute leukemias” refer to ALL. “Chronic leukemias” refer to CLL.

“Acute Lymphoblastic Leukemia (ALL)” refers to a blood cancer, particularly a cancer affecting the white bloodcells, and is characterized by hyperproliferation of lymphoblasts. In ALL, malignant, immature white blood cells continuously multiply and are overproduced in the bone marrow. ALL cells crowd out normal cells in the bone marrow and may metastasize to other organs. ALL is also known as acute lymphocytic leukemia and acute childhood leukemia. ALL includes B-cell acute lymphoblastic leukemia (B-ALL), and T-cell acute lymphoblastic leukemia (T-ALL).

“Chronic lymphocytic (or lymphoid) leukemia (CLL)” refers to a blood cancer affecting B cell lymphocytes. B cells originate in the bone marrow and develop in the lymph nodes. In CLL, the B cells grow in an uncontrolled manner and accumulate in the bone marrow and blood, wherein they crowd out healthy blood cells. As the disease advances, CLL results in swollen lymph nodes, spleen, and liver.

““Multiple myeloma (MM)” refers to a blood cancer having clonal B cell malignancy characterized by the accumulation of neoplastic plasma cells in the bone marrow. There are several types of multiple myeloma, including smoldering multiple myeloma (SMM), plasma cell leukemia, nonsecretory myeloma, osteosclerotic myeloma (POEMS syndrome), solitary plasmacytoma (also called solitary myeloma of the bone), and extramedullary plasmacytoma.

In one embodiment of the invention, the patient is afflicted with lung cancer. In the present invention, the term “lung cancer” means a malignant tumor occurring in a lung, and histologically includes lung adenocarcinoma, squamous cell carcinoma, non-small cell lung cancer (NSCLC), and small cell lung cancer (SCLC). In one embodiment of the invention, the lung cancer is a small cell lung cancer.

In one embodiment of the invention, the patient is afflicted with osteosarcoma. The term “osteosarcoma” refers to malignant tumor of bone or soft parts that arises from bone-forming mesenchymal cells.

In one embodiment of the invention, the patient is afflicted with an allergic disorder that is typically treated with GCs. As used herein, the terms “allergic disorder” or “allergic condition” refers to an abnormal biological function characterized by either an increased responsiveness of the trachea and bronchi to various stimuli or by a disorder involving inflammation at these or other sites in response to allergen exposure. Examples of allergic disorders include, but are not limited to, asthma, atopic dermatitis, bronchoconstriction, chronic airway inflammation, allergic contact dermatitis, eczema, food allergy, hay fever, hyper-IgE syndrome, rhinitis, and allergic urticaria.

In one embodiment of the invention, the patient is afflicted with an autoimmune disease that is typically treated with GCs. As used herein, the term “autoimmune disease” means a disease resulting from an immune response against a self tissue or tissue component, including both self antibody responses and cell-mediated responses. Such autoimmune diseases include, for example, type I diabetes mellitus (T1 D), Crohn's disease, ulcerative colitis, myasthenia gravis, vitiligo, Graves' disease, Hashimoto's disease, Addison's disease, autoimmune gastritis, autoimmune hepatitis, rheumatoid disease, systemic lupus erythematosus, progressive systemic sclerosis and variants, polymyositis, dermatomyositis, pernicious anemia including some of autoimmunegastritis, primary biliary cirrhosis, autoimmune thrombocytopenia, Sjogren's syndrome, multiple sclerosis and psoriasis.

### Pharmaceutical Composition

As used herein, the phrases “agent” and “pharmaceutical composition,” and the like, refer to the combination of one or more active agents, for example, one or more of the presently described miRNAs, and optionally one or more excipients, that is administered to a patient in need of treatment, and can be in any desired form, including for example, in the form of a solution, an aqueous solution, an emulsion, and a suspension.

Pharmaceutical compositions containing the presently described miRNAs as the active ingredient can be prepared according to conventional pharmaceutical compounding techniques. See, for example, Remington's Pharmaceutical Sciences, 18th Ed., Mack Publishing Co., Easton, Pa. (1990). See also, Remington: The Science and Practice of Pharmacy, 21st Ed., Lippincott Williams & Wilkins, Philadelphia, Pa. (2005). Suitable formulations can include, but are not limited to, injectable formulations including for example, solutions, emulsions, and suspensions. The compositions contemplated herein may take the form of solutions, suspensions, emulsions, combinations thereof, or any other pharmaceutical dosage form as would commonly be known in the art.

As used herein, the terms “administering”, “administration” and like terms refer to any method which, in sound medical practice, delivers a composition containing an active agent to a subject in such a manner as to provide a therapeutic effect. One aspect of the present subject matter provides for oral administration of a therapeutically effective amount of a composition of the present subject matter to a patient in need thereof. Other suitable routes of administration can include parenteral, subcutaneous, intravenous, intramuscular, or intraperitoneal. The dosage administered will be dependent upon the age, health, and weight of the recipient, kind of concurrent treatment, if any, frequency of treatment, and the nature of the effect desired.

As used herein, the term “carrier,” “excipient,” or “adjuvant” refers to any component of a pharmaceutical composition that is not the active agent.

As used herein, the term “pharmaceutically acceptable carrier” refers to a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present.

Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO.

These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety.

The presently described miRNAs may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

The term “purified” does not require the material to be present in a form exhibiting absolute purity, exclusive of the presence of other compounds. Rather, it is a relative definition. A peptide is in the “purified” state after purification of the starting material or of the natural material by at least one order of magnitude, 2 or 3, or 4 or 5 orders of magnitude.

The term “substantially free of naturally-associated host cell components” describes a peptide or other material which is separated from the native contaminants which accompany it in its natural host cell state. Thus, a peptide which is chemically synthesized or synthesized in a cellular system different from the host cell from which it naturally originates will be free from its naturally-associated host cell components.

As used herein, the term “substantially pure” describes a peptide or other material which has been separated from its native contaminants. Typically, a monomeric peptide is substantially pure when at least about 60 to 75% of a sample exhibits a single peptide backbone. Minor variants or chemical modifications typically share the same peptide sequence. A substantially pure peptide can comprise over about 85 to 90% of a peptide sample, and can be over 95% pure, over 97% pure, or over about 99% pure. Purity can be measured on a polyacrylamide gel, with homogeneity determined by staining. Alternatively, for certain purposes high resolution may be necessary and HPLC or a similar means for purification can be used. For most purposes, a simple chromatography column or polyacrylamide gel can be used to determine purity.

### Kits

In another aspect, the present invention provides kits suitable for assaying a plurality of miRNAs expression levels in a sample obtained from a patient. In another embodiment, there is provided a diagnostic kit comprising means for determining the expression level of a plurality of miRNAs selected from the group consisting of miR103, miR30e, miR30d, or combinations thereof. In another embodiment, the plurality of miRNAs further comprises miR15b. In another embodiment, the plurality of miRNAs further comprises miR16.

In another embodiment, the kit comprises miRNA hybridization or amplification reagents; and at least one probe or amplification primer specific for each member selected from the plurality of miRNAs. In some embodiments, the kit further comprises means for collecting a sample (e.g., blood) from a patient. In another embodiment, the diagnostic kit further comprises instructions for performing the necessary steps for determining miRNAs expression levels, e.g., in a sample obtained from a patient. In another embodiment, the kit further comprises reagents for flow cytometric analysis. In another embodiment, the kit further comprises means for detecting CD markers.

In some embodiment, determining miRNAs expression levels is utilized for evaluating the sensitivity of the patient to treatment with a glucocorticoid.

### Hybridization Assays

Detection of a nucleic acid of interest in a biological sample (e.g., miRNA) may optionally be effected by hybridization-based assays using an oligonucleotide probe. Traditional hybridization assays include PCR, reverse-transcriptase PCR, real-time PCR, RNase protection, in-situ hybridization, primer extension, dot or slot blots (RNA), and Northern blots (i.e., for RNA detection). More recently, PNAs have been described (Nielsen et al. 1999, Current Opin. Biotechnol. 10:71-75). other detection methods include kits containing probes on a dipstick setup and the like.

The term “probe” refers to a labeled or unlabeled oligonucleotide capable of selectively hybridizing to a target or template nucleic acid under suitable conditions. Typically, a probe is sufficiently complementary to a specific target sequence contained in a nucleic acid sample to form a stable hybridization duplex with the target sequence under a selected hybridization condition, such as, but not limited to, a stringent hybridization condition. A hybridization assay carried out using the probe under sufficiently stringent hybridization conditions permits the selective detection of a specific target sequence. For use in a hybridization assay for the discrimination of single nucleotide differences in sequence, the hybridizing region is typically from about 8 to about 100 nucleotides in length. Although the hybridizing region generally refers to the entire oligonucleotide, the probe may include additional nucleotide sequences that function, for example, as linker binding sites to provide a site for attaching the probe sequence to a solid support or the like, as sites for hybridization of other oligonucleotides, as restriction enzymes sites or binding sites for other nucleic acid binding enzymes, etc. In certain embodiments, a probe of the invention is included in a nucleic acid that comprises one or more labels (e.g., a reporter dye, a quencher moiety, a fluorescent labeling, etc.), such as a 5′-nuclease probe, a FRET probe, a molecular beacon, or the like, which can also be utilized to detect hybridization between the probe and target nucleic acids in a sample. In some embodiments, the hybridizing region of the probe is completely complementary to the target sequence. However, in general, complete complementarity is not necessary (i.e., nucleic acids can be partially complementary to one another); stable duplexes may contain mismatched bases or unmatched bases. Modification of the stringent conditions may be necessary to permit a stable hybridization duplex with one or more base pair mismatches or unmatched bases. Sambrook et al., Molecular Cloning: A Laboratory Manual, 3rd Ed., Cold Spring Harbor Laboratory Press, Cold Spring Harbor, N.Y. (2001), which is incorporated by reference, provides guidance for suitable modification. Stability of the target/probe duplex depends on a number of variables including length of the oligonucleotide, base composition and sequence of the oligonucleotide, temperature, and ionic conditions. One of skill in the art will recognize that, in general, the exact complement of a given probe is similarly useful as a probe. One of skill in the art will also recognize that, in certain embodiments, probe nucleic acids can also be used as primer nucleic acids. Exemplary probe nucleic acids include 5′-nuclease probes, molecular beacons, among many others known to persons of skill in the art.

As used herein, “hybridization” refers to a reaction in which at least one polynucleotide reacts to form a complex that is stabilized via hydrogen bonding between the bases of the nucleotide residues. The hydrogen bonding may occur by. Watson-Crick base pairing, in any other sequence-specific manner. A hybridization reaction may constitute a step in a more extensive process, such as the initiation of a PCR reaction.

Hybridization reactions can be performed under conditions of different stringency. Under stringent conditions, nucleic acid molecules at least 60%, 65%, 70%, 75% identical to each other remain hybridized to each other. A non-limiting example of highly stringent hybridization conditions is hybridization in 6*sodium chloride/sodium citrate (SSC) at approximately 45° C., followed by one or more washes in 0.2*SSC and 0.1% SDS at 50° C., at 55° C., or at about 60° C. or more.

When hybridization occurs in an anti-parallel configuration between two single-stranded polynucleotides, those polynucleotides are described as complementary.

Hybridization based assays which allow the detection of a biomarker of interest in a biological sample rely on the use of probe(s) which can be 10, 15, 20, or 30 to 100 nucleotides long optionally from 10 to 50, or from 40 to 50 nucleotides long.

Thus, the polynucleotides of the biomarkers of the invention, according to at least some embodiments, are optionally hybridizable with any of the herein described nucleic acid sequences under moderate to stringent hybridization conditions.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample.

Probes can be labeled according to numerous well known methods. Non-limiting examples of detectable markers include ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe.

For example, oligonucleotides according to at least some embodiments of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, Fluor X (Amersham) and others (e.g., Kricka et al. (1992), Academic Press San Diego, Calif.) can be attached to the oligonucleotides. Preferably, detection of the biomarkers of the invention is achieved by using TaqMan assays, preferably by using combined reporter and quencher molecules (Roche Molecular Systems inc.).

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods.

As commonly known, radioactive nucleotides can be incorporated into probes of the invention by several methods. Non-limiting examples of radioactive labels include 3H, 14C, 32P, P and 35S.

Those skilled in the art will appreciate that wash steps may be employed to wash away excess target polynucleotide or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNAse A prior to hybridization, to assess false hybridization.

Probes of the invention can be utilized with naturally occurring sugar-phosphate backbones as well as modified backbones including phosphorothioates, dithionates, alkyl phosphonates and a-nucleotides and the like. Probes of the invention can be constructed of either ribonucleic acid (RNA) or deoxyribonucleic acid (DNA).

### Flow Cytometry

Flow cytometry is described in the extensive literature in this field, including, for example, Flow Cytometry Protocols, Methods in Molecular Biology No. 91, Humana Press (1997); and Shapiro, Practical FlowCytometry, 4th ed., Wiley-Liss (2003); incorporated herein by reference. Flow cytometry is a technique used to analyze multiple phenotypic and functional parameters simultaneously within a single cell or a population of cells. Cell components are fluorescently labeled, and then excited by the laser to emit light at varying wavelengths. Different components, can be labeled with different fluorescents, therefore fluorescence can be measured to simultaneously determine various components of single cells. In a non-limiting example, in order to analyze the presence of miR-103 in B-ALL cell population, CD20 protein (typically expressed on the surface of B-ALL cells), can be labeled with a specific fluorescent antibody, and miR-103 can be hybridized to a specific fluorescent probe. Due to the double labeling, flow cytometry analysis will demonstrate the presence of miR-103 in B-ALL cells population. In specific embodiments, said determination is on a single cell level. In yet another non-limiting example, in order to analyze the presence of miR-103 in T-ALL cell population, CD3 protein (typically expressed on the surface of T-ALL cells), can be labeled with a specific fluorescent antibody, and miR-103 can be hybridized to a specific fluorescent probe. Due to the double labeling, flow cytometry analysis will demonstrate the presence of miR-103 in T-ALL cells population, such in a single cell resolution.

### Fluorescence In Situ Hybridization (FISH)

An additional NAT test known in the art is Fluorescence In Situ Hybridization (FISH). FISH uses fluorescent single-stranded DNA or RNA probes which are complementary to the nucleotide sequences that are under examination (genes, chromosomes or RNA). These probes hybridize with the complementary nucleotide and allow the identification of the chromosomal location of genomic sequences of DNA or RNA.

Detection of a nucleic acid of interest in a biological sample may also optionally be effected by NAT-based assays, which involve nucleic acid amplification technology, such as PCR for example (or variations thereof such as real-time PCR for example).

As used herein, a “primer” defines an oligonucleotide which is capable of annealing to (hybridizing with) a target sequence, thereby creating a double stranded region which can serve as an initiation point for DNA synthesis under suitable conditions. Although other primer nucleic acid lengths are optionally utilized, they typically comprise hybridizing regions that range from about 8 to about 100 nucleotides in length. Short primer nucleic acids generally utilize cooler temperatures to form sufficiently stable hybrid complexes with template nucleic acids. A primer nucleic acid that is at least partially complementary to a subsequence of a template nucleic acid is typically sufficient to hybridize with the template for extension to occur. A primer nucleic acid can be labeled (e.g., a SCORPION primer, etc.), if desired, by incorporating a label detectable by, e.g., spectroscopic, photochemical, biochemical, immunochemical, chemical, or other techniques. To illustrate, useful labels include radioisotopes, fluorescent dyes, electron-dense reagents, enzymes (as commonly used in ELISAs), biotin, or haptens and proteins for which antisera or monoclonal antibodies are available. Many of these and other labels are described further herein and/or otherwise known in the art. One of skill in the art will recognize that, in certain embodiments, primer nucleic acids can also be used as probe nucleic acids.

Amplification of a selected, or target, nucleic acid sequence may be carried out by a number of suitable methods (e.g., Kwoh et al., 1990, Am. Biotechnol. Lab. 8:14). Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the q3 replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra).

The terminology “amplification pair” (or “primer pair”) refers herein to a pair of oligonucleotides according to at least some embodiments of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

In one particular embodiment, amplification of a nucleic acid sample from a patient is amplified under conditions which favor the amplification of the most abundant differentially expressed nucleic acid. In one embodiment, RT-PCR is carried out on an RNA sample from a patient under conditions which favor the amplification of the most abundant RNA. In another embodiment, the amplification of the differentially expressed nucleic acids is carried out simultaneously. It will be realized by a person skilled in the art that such methods could be adapted for the detection of differentially expressed proteins instead of differentially expressed nucleic acid sequences.

In particular embodiments, TagMan® microRNA assay may be used for evaluating the expression levels of the microRNAs panel of the invention. Non limiting example for evaluating the expression level of the microRNAs of the invention is TagMan® microRNA assay ID 000439 for evaluating miR103.

The nucleic acid (e.g., RNA) for practicing the present invention may be obtained according to well known methods.

Oligonucleotide primers according to at least some embodiments of the present invention may be of any suitable length, depending on the particular assay format and the particular needs and targeted genomes employed. Optionally, the oligonucleotide primers are at least 12 nucleotides in length, preferably between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence (Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

The polymerase chain reaction and other nucleic acid amplification reactions are well known in the art. The pair of oligonucleotides according to this aspect of the present invention are preferably selected to have compatible melting temperatures (Tm), e.g., melting temperatures which differ by less than that 7° C., preferably less than 5° C., more preferably less than 4° C., most preferably less than 3° C., ideally between 3° C. and 0° C.

### Polymerase Chain Reaction (PCR)

The polymerase chain reaction (PCR), as described in U.S. Pat. Nos. 4,683,195 and 4,683,202 to Mullis and Mullis et al., is a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, they are said to be “PCR-amplified”.

Any concentration ranges, percentage range, or ratio range recited herein are to be understood to include concentrations, percentages or ratios of any integer within that range and fractions thereof, such as one tenth and one hundredth of an integer, unless otherwise indicated.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Various embodiments and aspects of the present invention as delineated hereinabove and as claimed in the claims section below find experimental support in the following examples.

Reference is now made to the following examples, which together with the above descriptions illustrate some embodiments of the invention in a non-limiting fashion.

### Material and Methods:

Cells and reagents:

Cell lines models of human T-cell acute lymphocytic leukemia (T-ALL) and Burkitt's lymphoma (BL), including the glucocorticoid (GC) sensitive cells represented by CEM-C7H2 (T-ALL, the sensitive sub-clone of CCRF-CEM), LOUCY (T-ALL) and DAUDI (BL), and the GC resistant cells represented by MOLT-4 (T-ALL), CUTLL (T-ALL), BJAB (BL) and SUD-136 (BL) were used. At least three kinds of cells were used in each type of cancer in order to minimize cell type specific variables. In addition, two types of malignancies were examined, T-ALL to represent leukemia behavior and BL to represent lymphoma behavior. Bone marrow cells were collected from normal donors and ALL patients. All experiments with human cells were approved by an institutional Helsinki committee and the Ministry of Health. Dexamethasone (DEX), RU486, Propidium iodide (PI), puromycin and Tri-reagent were purchased from sigma and TransIT-LT1 from Mirus.

Deep Sequencing:

RNA of cell samples was extracted by Tri-reagent. 1 μg of each sample was sequenced on an Illumina Genome Analyzer as described by Son et al, Curr Protoc Microbiol; Chapter 1: Unit 1E 4. Data analysis was performed by a miRNAkey application Ronen et al., Bioinformatics; 26(20):2615-2616.

Plasmids:

pLKO.1 control, sh-scrumble, SinGFP controls miR-K2 and Sp-UL112 plasmids were used. Sh-c-Myc and sh-BIM with their control plasmids were purchased from Openbiosystem and miR-17 92a plasmids from GeneCopoeia.

miRNA Overexpression:

The miRNA oligomers used in this study are outlined in Table 3. Oligos were annealed, phosphorylated and inserted into a pTER plasmid in order to connect the overexpressed miRNA to the U6 promoter. The reconstructed pTER was sequenced by the H1 primer 5′ CGCTGACGTCATCAACCCGC 3′. The miRNA/U6 construct was excised and inserted into a Sin-GFP plasmid.

TABLE 3
The oligonucleotide sequence used to
construct miRNAs overexpression plasmids.

| | | | SEQ |
|---|---|---|---|
| Oligos to mi-RNAs over-expression constructs | | | ID NO: |
| 5′GATCCCCAGCAGCATTGTACAGGGCTATGATTCAAGAGATC | FW | hsa-miR-103-5p | 41 |
| ATAGCCCTGTACAATGCTGCTTTTTTGGAAA 3′ | | | |
| 5′AGCTTTTCCAAAAAAGCAGCATTGTACAGGGCTATGATCTC | RV | | 42 |
| TTGAATCATAGCCCTGTACAATGCTGCTGGG 3′ | | | |
| 5′GATCCCCTGTAAACATCCTTGACTGGAAGTTCAAGAGACTT | FW | hsa-miR-30e-5p | 43 |
| CCAGTCAAGGATGTTTACATTTTTGGAAA 3′ | | | |
| 5′AGCTTTTCCAAAAATGTAAACATCCTTGACTGGAAGTCTCT | RV | | 44 |
| TGAACTTCCAGTCAAGGATGTTTACAGGG 3′ | | | |
| 5′GATCCCCTGTAAACATCCCCGACTGGAAGTTCAAGAGACTT | FW | hsa-miR-30d-5p | 45 |
| CCAGTCGGGGATGTTTACATTTTTGGAAA 3′ | | | |
| 5′AGCTTTTCCAAAAATGTAAACATCCCCGACTGGAAGTCTCT | RV | | 46 |
| TGAACTTCCAGTCGGGGATGTTTACAGGG 3′ | | | |
| 5′GATCCCCTGTGCAAATCCATGCAAAACTGATTCAAGAGATC | FW | hsa-miR-19b-5p | 47 |
| AGTTTTGCATGGATTTGCACATTTTTGGAAA 3′ | | | |
| 5′AGCTTTTCCAAAAATGTGCAAATCCATGCAAAACTGATCTC | RV | | 48 |
| TTGAATCAGTTTTGCATGGATTTGCACAGGG 3′ | | | |
| 5′GATCCCCTAAAGTGCTTATAGTGCAGGTAGTTCAAGAGACT | FW | hsa-miR-20a-5p | 49 |
| ACCTGCACTATAAGCACTTTATTTTTGGAAA 3′ | | | |
| 5′AGCTTTTCCAAAAATAAAGTGCTTATAGTGCAGGTAGTCTC | RV | | 50 |
| TTGAACTACCTGCACTATAAGCACTTTAGGG 3′ | | | |
| 5′GATCCCCTGTCAGTTTGTCAAATACCCCATTCAAGAGATGG | FW | hsa-miR-223-5P | 51 |
| GGTATTTGACAAACTGACATTTTTGGAAA 3′ | | | |
| 5′AGCTTTTCCAAAAATGTCAGTTTGTCAAATACCCCATCTCTT | RT | | 52 |
| GAATGGGGTATTTGACAAACTGACAGGG 3′ | | | |
| 5′GATCCCCCGAATCATTATTTGCTGCTCTATTCAAGAGATAG | FW | hsa-miR-15b-5p | 53 |
| AGCAGCAAATAATGATTCGTTTTTGGAAA 3′ | | | |
| 5′AGCTTTTCCAAAAACGAATCATTATTTGCTGCTCTATCTCTT | RV | | 54 |
| GAATAGAGCAGCAAATAATGATTCG GGG 3′ | | | |
| 5′AGCTTTTCCAAAAATAGCAGCACGTAAATATTGGCGTCTCT | FW | hsa-miR-16-5p | 55 |
| CAATATTTACGTGCTGCTATTTTTGGAAA 3′ | | | |
| 5′AGCTTTTCCAAAAATAGCAGCACGTAAATATTGGCGTCTCT | RV | | 56 |
| TGAACGCCAATATTTACGTGCTGCTAGGG 3′ | | | |

miRNA Sponge:

Sponge assay is a method, known in the art, for miRNAs down-regulation. The oligonucleotides of miRNA sponges are outlined in Table 4. These oligos were annealed, phosphorylated and inserted one after the other into a pBSII plasmid. The reconstructed pBSII plasmid was sequenced by the T7 primer 5′ TAATACGACTCACTATAGGG 3′. Consequently, the joint pairs A and B were excised and inserted into a Sin-GFP plasmid.

TABLE 4
The oligonucleotide sequence used to
construct miR-103 sponge plasmid.

| | | | | SEQ |
|---|---|---|---|---|
| Oligos to mi-RNAs sponge construct | | | | ID NO: |
| 5′TCGACTCATAGCCCTGTGTATGCTGCTAGAGTCATAGCCCTGT | FW | A | hsa-miR-103-5p | 57 |
| GTATGCTG CT AGAG TCATAGCCCT GTGTATGCTG CT A 3′ | | | | |
| 5′AGCTTAGCAGCATACACAGGGCTATGACTCTAGCAGCATA | RV | | | 58 |
| CACAGGGCTA TGACTCTAGC AGCATACACA GGGCTATGAG 3′ | | | | |
| 5′AGCTTTCATAGCCCTGTGTATGCTGCTAGAG | FW | B | | 59 |
| TCATAGCCCTGTGTATGCTGCTAGAGTCATAGCCCT | | | | |
| GTGTATGCTG CT G 3′ | | | | |
| 5′AATTCAGCAGCATACACAGGGCTATGACTCTAGCAGCATA | RV | | | 60 |
| CACAGGGCTA TGACTCTAGC AGCATACACA GGGCTATGAA 3′ | | | | |

Stable Transfection:

Lentivirus particles were prepared in 293T cells with the pCMV/Δ8.91 packaging vector and pMD2-VSV-G envelope construct by using TranslT-LT1 transfection reagent. Lentivirus supernatant was collected after 48 hrs and incubated with cells for 24 hrs. pLKO.1, pEZX and pGIPZ transfected cells were enriched by selection in 1-4 μg/ml puromycin.

Apoptosis:

Cells were treated with 100 nM Dex for 72 hrs. Percent apoptotic cells was determined by PI-uptake as measured in a FACS Calibur flow cytometer (BD).

Cellular Proliferation:

A BrdU Cell Proliferation Assay kit (Exalpha Biological, Inc) or a BrdU Flow Kit (BD Pharmingen) were used to determine cellular proliferation.

Western Blots and Antibodies:

Total lysate of 10⁶ cells was prepared in 50 μl protein sample buffer (PSB) ×1.5. The following antibodies were used for immuno-blotting: anti-β-Catenin and anti-GR (BD Transduction Laboratories); anti-BIM (Calbichem); anti-CDK2, anti-c-Myb, anti-DVL1 and anti-c-Myc (Santa Cruz Biotechnology); anti-cycline E1 (Cell Signaling Technology); and anti-a-Tubulin (Sigma). Immune-blots images are presented by Image Lab 3.0 software. Specific bands were cropped by transferring images to Adobe Photoshop CS5.

RT-PCR:

Total RNA was isolated using miRNeasy mini kit (QIAGEN). For Syber green assay, precipitated RNA samples were treated with Poly A Tailing kit (Ambion). c-DNA was prepared by M-MLV reverse transcriptase (Invitrogen). q-PCR amplification of mRNA was operated by Platinum SYBER Green (Invitrogen) with the following primers: FKBP5 5′ TACTGATGAAGGTGCCAAGAACA 3′ and 5′ GTCTCCAATCATCGGCGTT 3′, SGK1 5′ CCTGCATTCACTGAACATCGT 3′ and 5′ TAAGGACAATGTGTCCCTGTGAAT 3′. For Taqman based PCR, c-DNA was prepared by Taqman MicroRNA Reverse Transcription Kit (Applied Biosystem). q-PCR was performed using TaqMan 2× Universal PCR Master Mix (Applied Biosystem).

Chromatin IP:

The EpiTech ChIP OneDay kit (Qiagen) was employed. IP antibodies used were: Rabbit-IgG (Qiagen), anti-GR (cell signaling) and anti-H3K4me1 (abcam). qRT-PCR was performed by syber green enzyme with the following primers: PANK3-GRE, GAPDH positive control and MYOD1 negative control (Qiagen). GR-GRE primers were planned as follow: Fw-5′ ATTCTTGTGCCTATGCAGACATTT 3′ and 5′ TGAATGCGTGCATATTCACACTA 3′. % Enrichment was calculated according to the formula: 2̂(C_{T} Mean(Input)−C_{T} Mean of (Interested fraction))*100.

Statistics:

Each experiment was repeated at least three times. p-value was calculated using the Fisher-Irwin test. p-values map: * p-Value<0.05, **p-Value<0.01 and ***p-Value<0.001. p-values below 0.05 is considered statistically significant.

miR-103 Expression in Patients Bone Marrow and Blood:

Blood and bone marrow (BM) samples of pediatric acute lymphoblastic leukemia (ALL) and chronic lymphoblastic leukemia (CLL) patients were collected from hospitals for testing, and the treatment and clinical status of these patients cross-checked in a blind manner. The outline of the study was to collect blood samples from ALL patients at diagnosis, incubate the blood with or without PRED overnight and then measure miR-103 expression levels. 11 BMs of ALL patients and 6 BMs of normal pediatric donors were analyzed. Furthermore, peripheral blood samples from 8 pediatric ALL patients and 1 CLL patient were collected. The CLL and 5 ALL samples were treated with PRED in vitro while from the additional 3 ALL patients blood was taken at diagnosis and at day 1 following PRED treatment.

### Example 1: GC Modulates miRNAs Expression

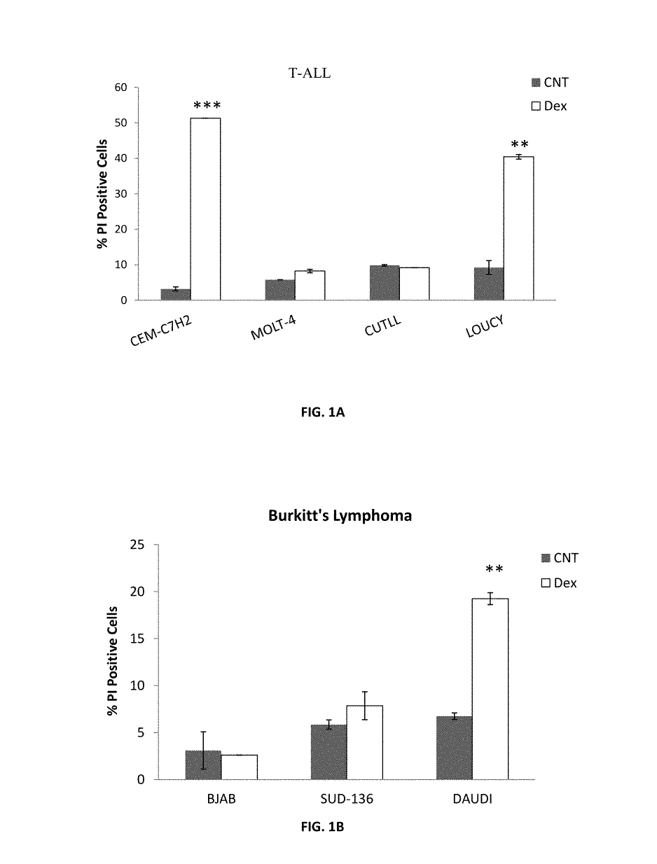

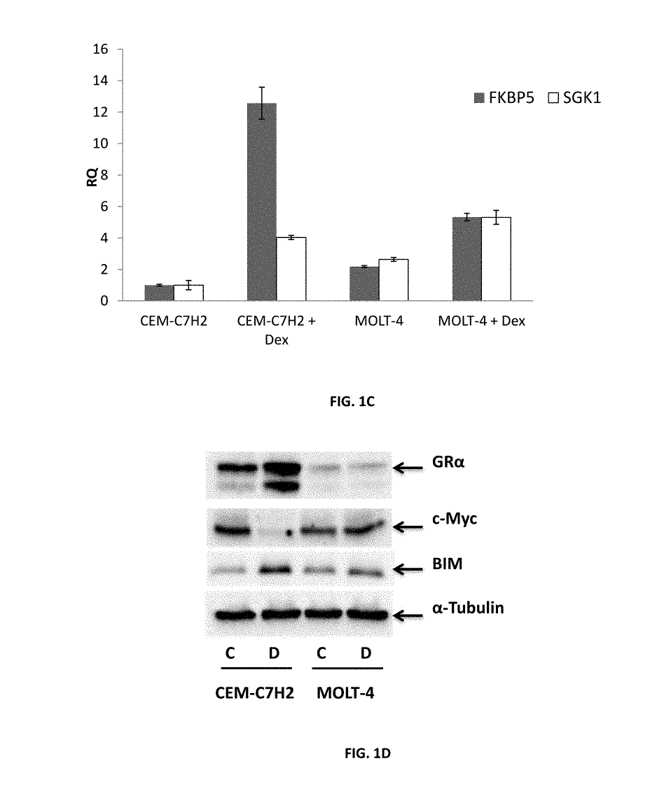

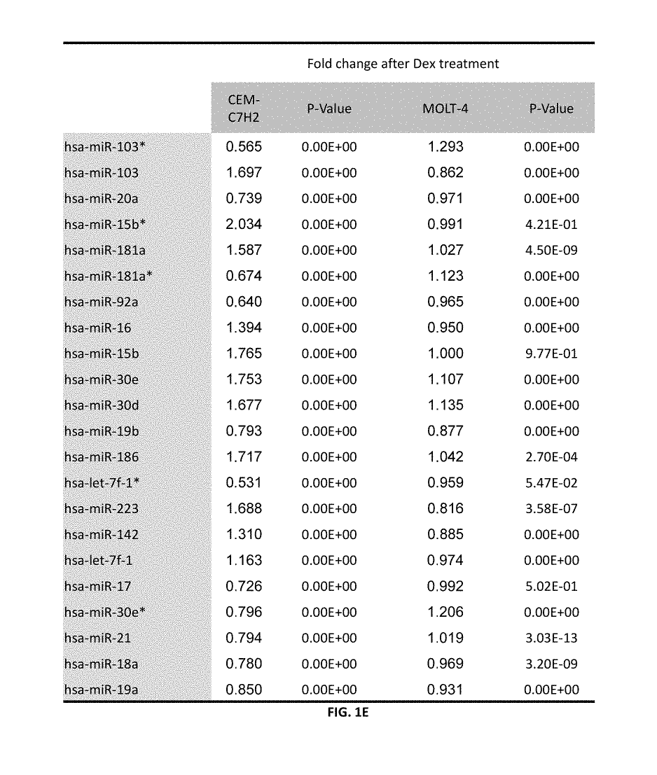

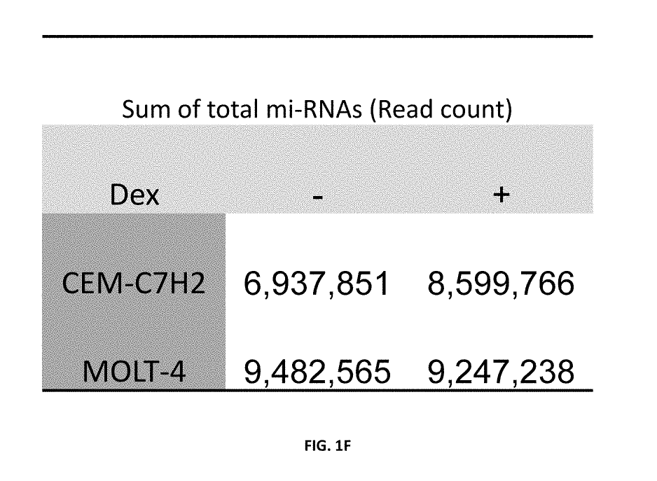

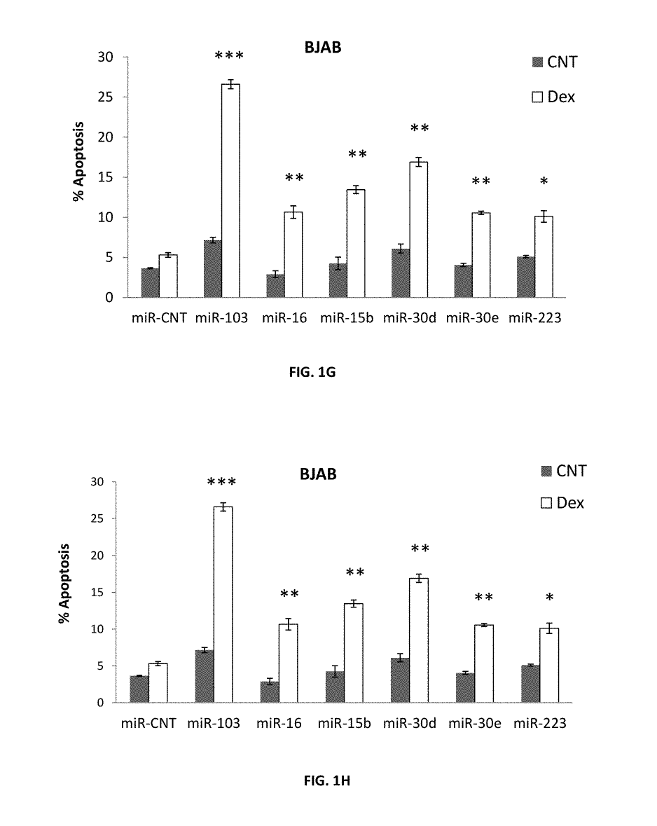

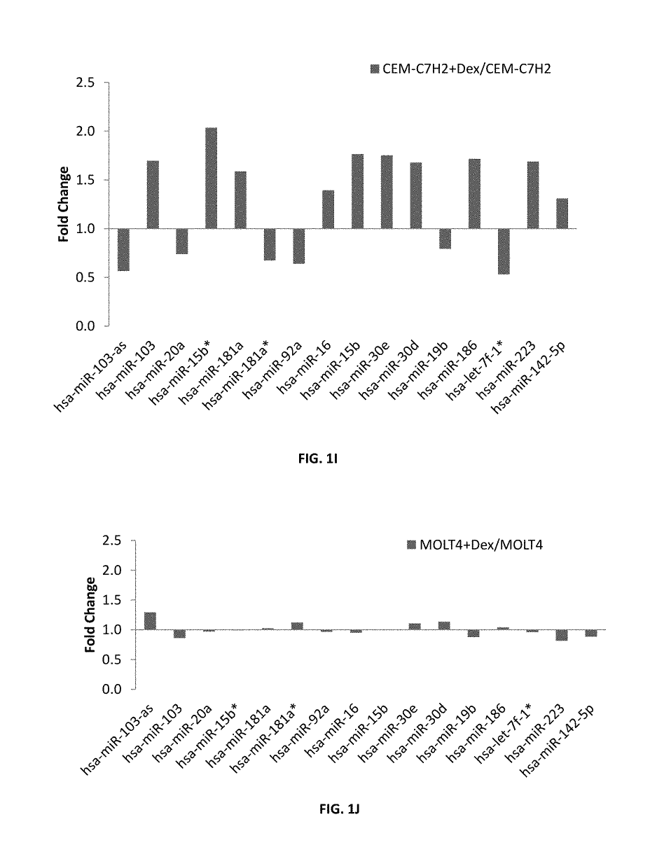

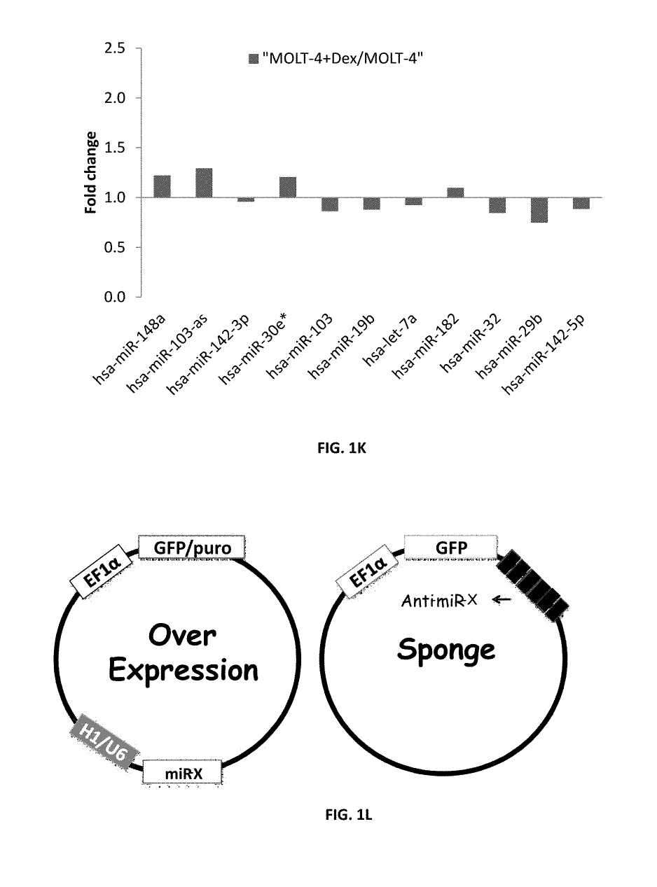

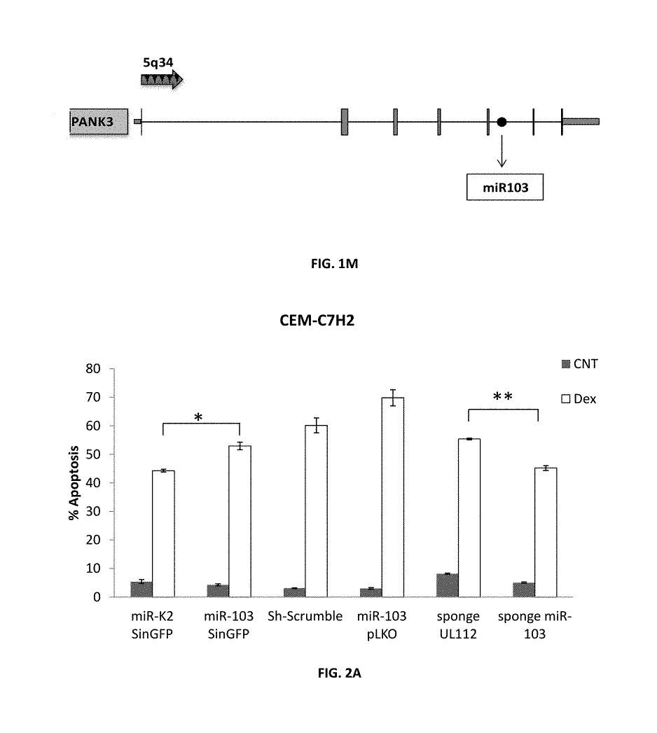

Glucocorticoid (GC) sensitive and resistant cells were treated with, the glucocorticoid dexamethasone (Dex) for 72 hours. Dex induced apoptosis of the sensitive cells, CEM-C7H2, LOUCY and DAUDI, while the GC resistant cells, MOLT-4, CUTLL, BJAB and SUD-136, survived (FIG. 1A-B). Consequently, CEM-C7H2 and MOLT-4 were chosen for further analysis. Both cells express functional GC receptor (GR) and display normal gene regulation following exposure to Dex, such as the up regulation of FKBP1 and SGK1 (FIG. 1C).

The effect of Dex on the level of consensus proteins which partake in the GC signal was examined. Results show that in CEM-C7H2, Dex induced upregulation of GR and BIM whereas c-Myc was downregulated (FIG. 1D). By contrast, in MOLT-4 these proteins were almost not affected (FIG. 1D).

To gain an insight into the molecular pathways regulating the expression of these proteins, CEM-C7H2 and MOLT-4 cells, treated or untreated with Dex, were subjected to deep sequencing of small RNAs. This analysis revealed 22 miRNAs that their expression levels were most significantly modulated by Dex in the sensitive CEM-C7H2 cells. The expression level of miR-103, miR-15b, miR-181a, has-let-7f, miR-16, miR-30e and miR-30d, miR-15b* (has-miR-15b-3p), miR-21, miR-186 and miR223 was elevated (FIGS. 1E and 1I). Wherein the expression level of miR-103* (has-miR-103-3p), miR-20a, miR-181a* (has-miR-181-3p), miR-92a, miR-17, miR-30e*, miR-19b, miR-18a, miR-19a. These miRNAs were not significantly modulated in Dex-treated MOLT-4 cells (FIGS. 1E and 1J).

Furthermore, total miRNAs expression in MOLT-4 was less significantly modulated following exposure to Dex compared with CEM-C7H2 (FIGS. 1F and 1K), indicating a qualitative difference between the two. Real time PCR (qRT-PCR) analysis validated the deep sequencing data.

### Example 2: miR-103 Confers GCIA

Next, the inventors examined which of Dex-regulated miRNAs play an essential role in GC induced apoptosis (GCIA) (FIG. 1L) In order to find, miRNAs that impose a GC-sensitive phenotype on otherwise GC-resistant cells, overexpression and sponge plasmids of miRNAs were constructed and expressed in GC resistant MOLT-4 and BJAB cells. Transfection with miR-103 was particularly effective in conferring GC sensitivity upon BJAB and MOLT-4 cells (FIG. 1G-H). This further validates the deep sequencing data, marking miR-103 as most significantly modulated upon Dex stimulation (FIG. 1E). Other miRNAs examined, such as miR-15b, miR-16, miR-30e and miR-30d were also found to sensitized BJAB and MOLT-4 cells to GCIA.

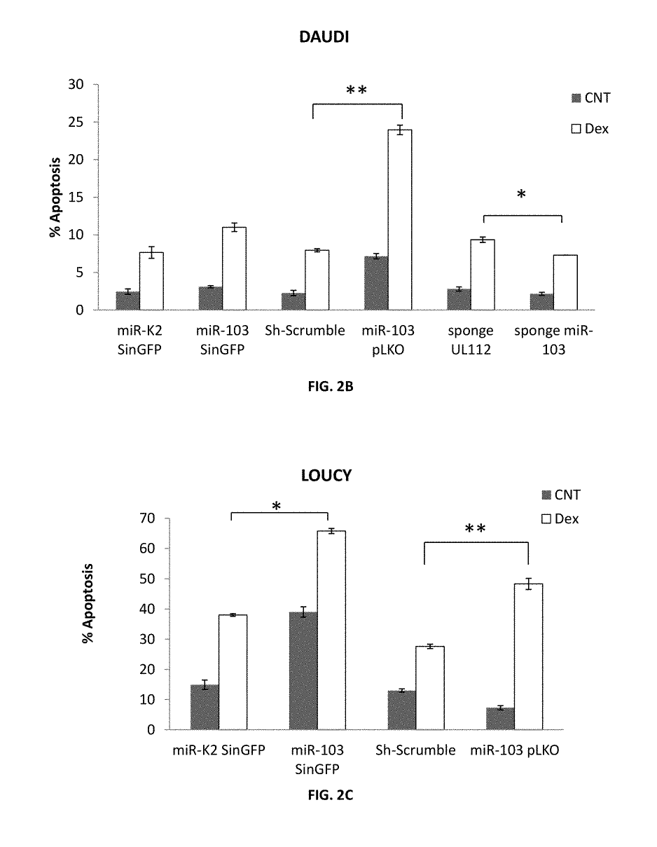

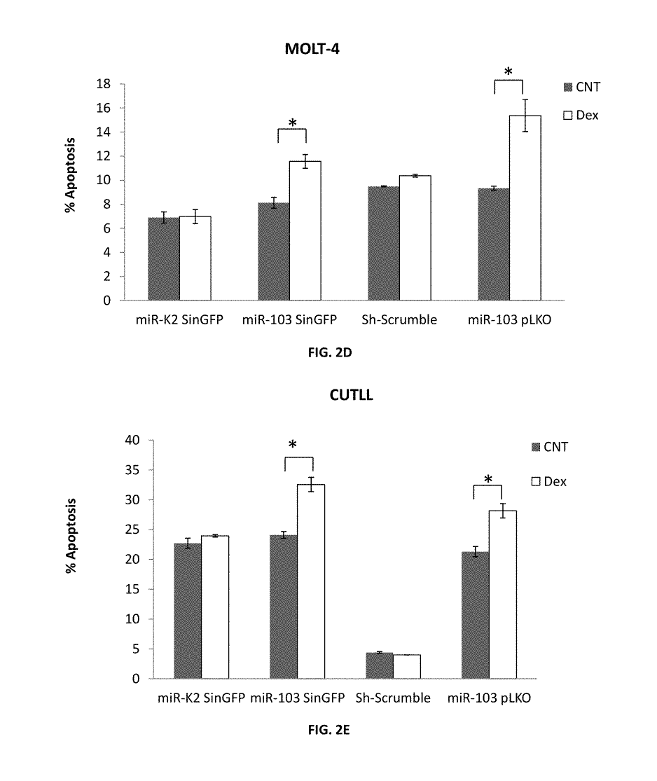

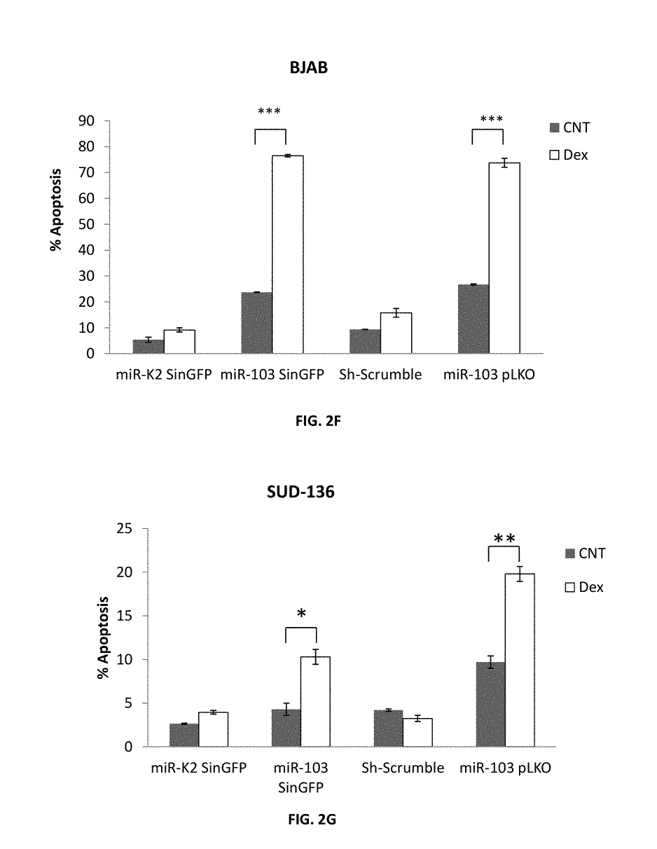

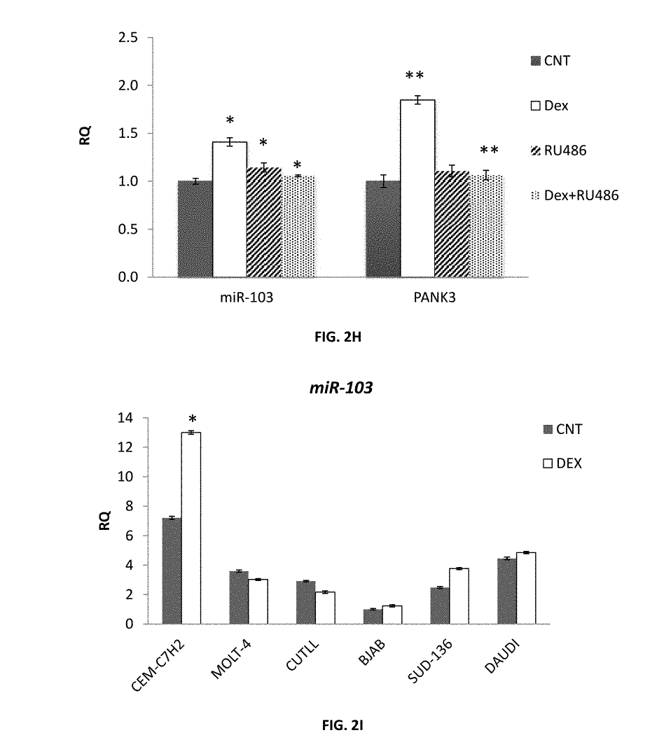

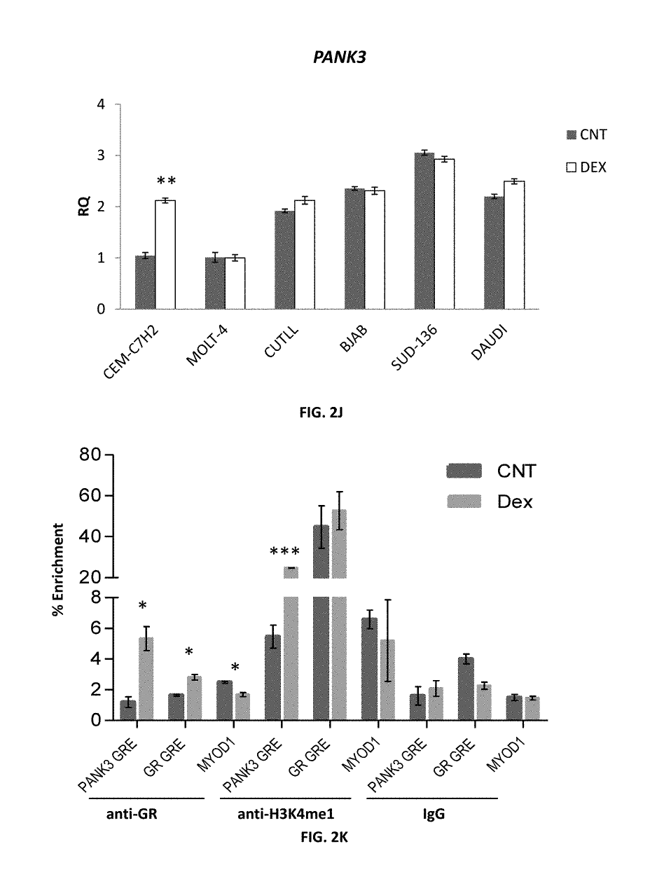

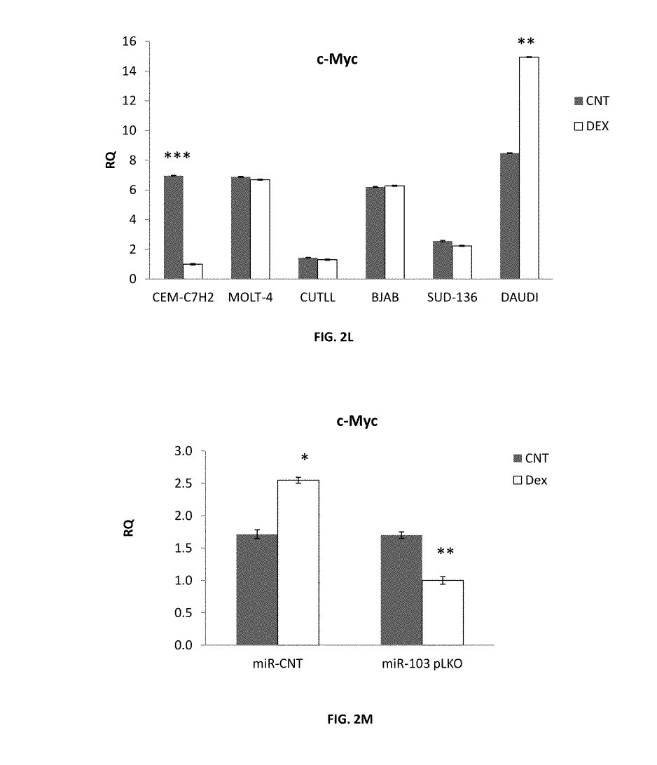

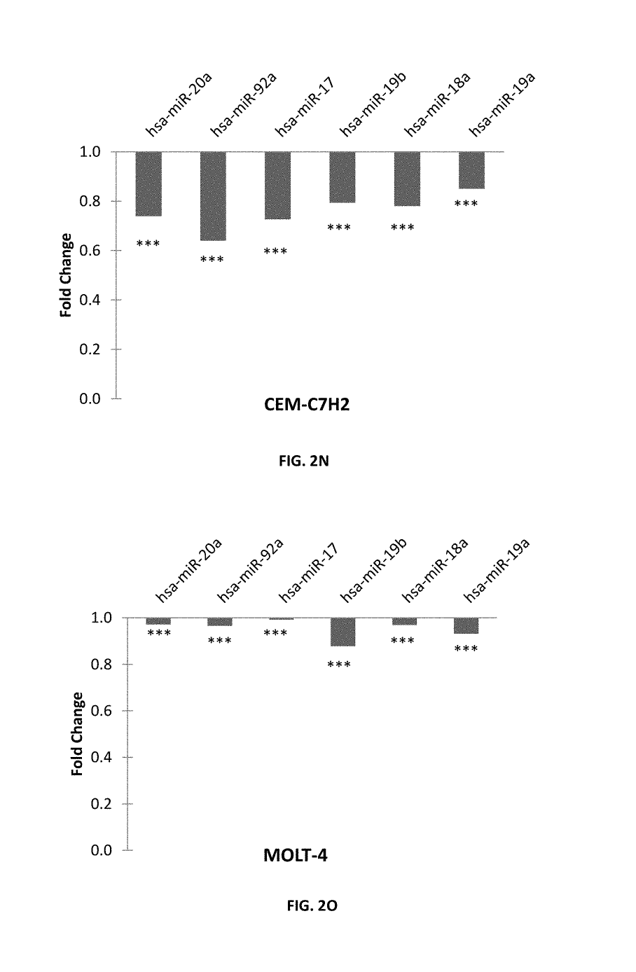

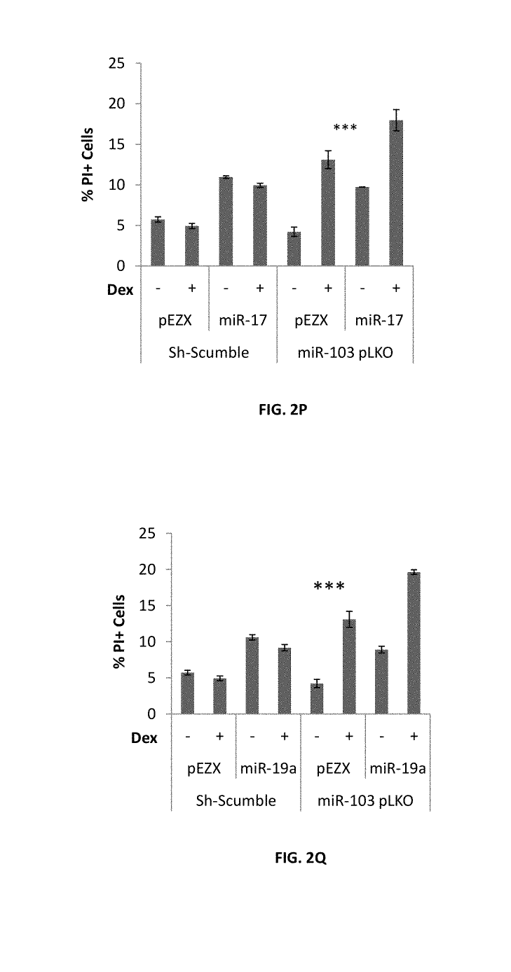

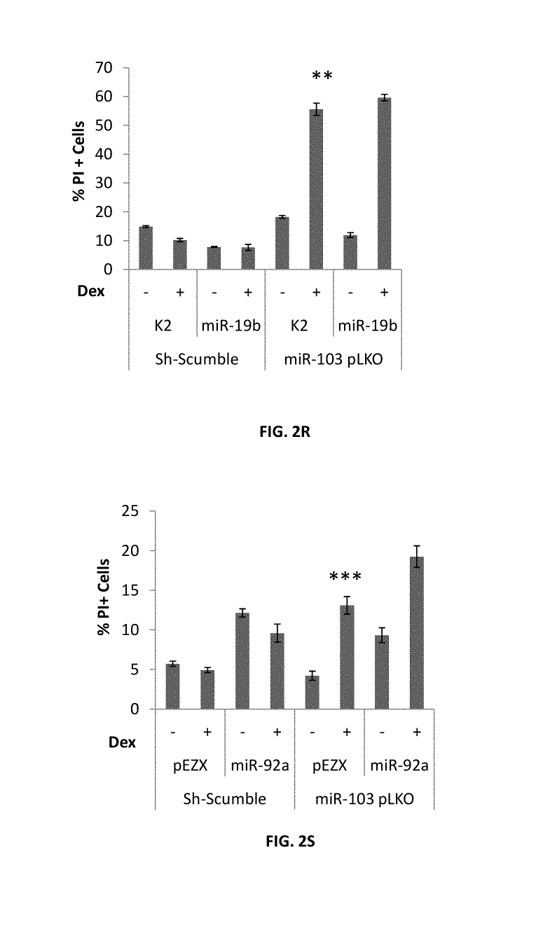

Consequently, the effect of miR-103 on GCIA was examined by transfection with concentrated miR-103 overexpression or sponge plasmids. In untreated cells, miR-103 overexpression had only a minor effect on apoptosis. However, when treated with Dex, resistant MOLT-4, CUTLL, BJAB and SUD136 cells overexpressing miR-103 underwent significant apoptosis (FIG. 2D-G). Moreover, GC sensitive CEM-C7H2, DAUDI and LOUCY cells overexpressing miR-103 became hyper-sensitive to GCIA (FIG. 2A-C). Furthermore, when miR-103 was sponged (down regulated) in CEM-C7H2 and DAUDI, a slight reduction in apoptosis was observed following treatment with Dex (FIG. 2A-B). These results indicate that miR-103 plays a pivotal role in GCIA. The efficacy of miR-103 absorption by the sponge could not be determined. Hence, the moderate reduction in GCIA can be attributed to partial absorption of miR-103 by the sponge.

Unexpectedly, elevating miR-103 expression in GC-resistant cells conferred sensitivity to GC-induced apoptosis. Vice versa, cellular miR-103 inhibition reduced GC-induced apoptosis in GC-sensitive cells.

### Example 3: miR-103 Expression in Cell Lines and Various Human Organs

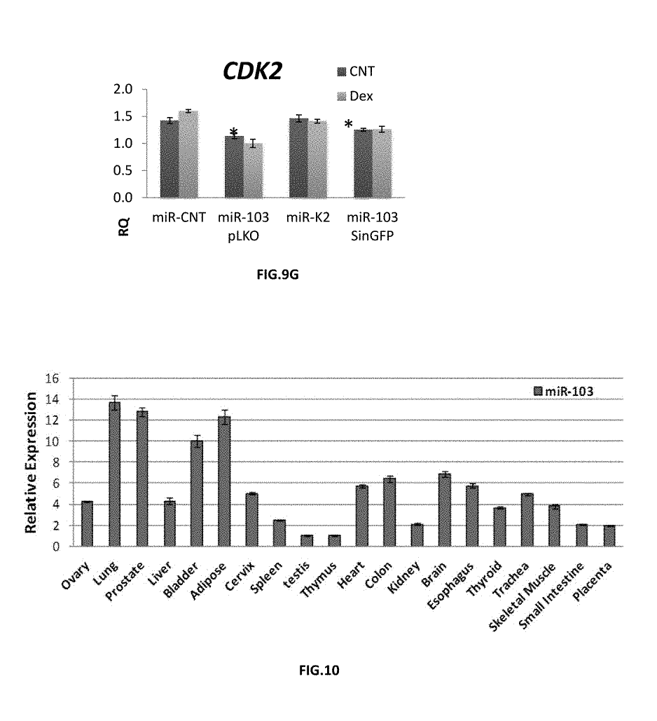

Examining miR-103 expression in a set of twenty human organs suggested that miR-103 is highly expressed in “resting” organs whereas its expression is low in “proliferating” organs, such as the thymus and spleen, where lymphocytes are stored (FIG. 10).

miR-103 resides within the fifth intron of the PANK3 gene (FIG. 1M). In order to investigate whether miR-103 and PANK3 are co-regulated, the relative quantification (RQ) of these two genes in untreated or Dex-treated cells was analyzed. As shown in FIG. 2I-J, there is no correlation between basal miR-103 and PANK3 levels. However, following Dex treatment, miR-103 and PANK3 were both upregulated in CEM-C7H2 but remained unchanged in other cells (FIG. 2I-J). Moreover, inhibition of GR by its inhibitor RU486, blocked both miR-103 and PANK3 upregulation (FIG. 2H). Even though DAUDI cells are moderately sensitive to GC, neither their miR-103 nor PANK3 expression was increased in response to Dex (FIG. 2I-J), explaining why DAUDI display only marginal response to Dex (FIG. 1A-B).

A GRE sequence was discovered in PANK3 promoter, about 10,000 base pairs upstream to PANK3 coding region. This domain (TGTGCAAACTATTCTT) is located at chr5:168017288-168017303 and is compatible with a consensus GRE. Next, chromatin immunoprecipitation (IP) assay was applied on CEM-C7H2 cells untreated or treated with Dex. Using an anti-GR antibody, PANK3-GRE was found to be enriched by approximately 5% in Dex-treated compared with untreated cells (FIG. 2K), indicating that upon activation, GR binds to GRE within PANK3 promoter. Furthermore, PANK3-GRE immunoprecipitated by anti-H3K4me1 was enriched nearly fivefold in Dex-treated cells (FIG. 2K), suggesting that PANK3-GRE is an enhancer. No Dex-induced PANK3-GRE enrichment by control IgG antibody was observed (FIG. 2K). As a positive control two bona fide GRE domains of the GR promoter were used. Indeed, GR IP in Dex-treated cells was followed by enrichment of GR-GRE while the negative control MYOD1 was not enriched (FIG. 2K).

### Example 4: miR-103 Downregulates c-Myc Expression

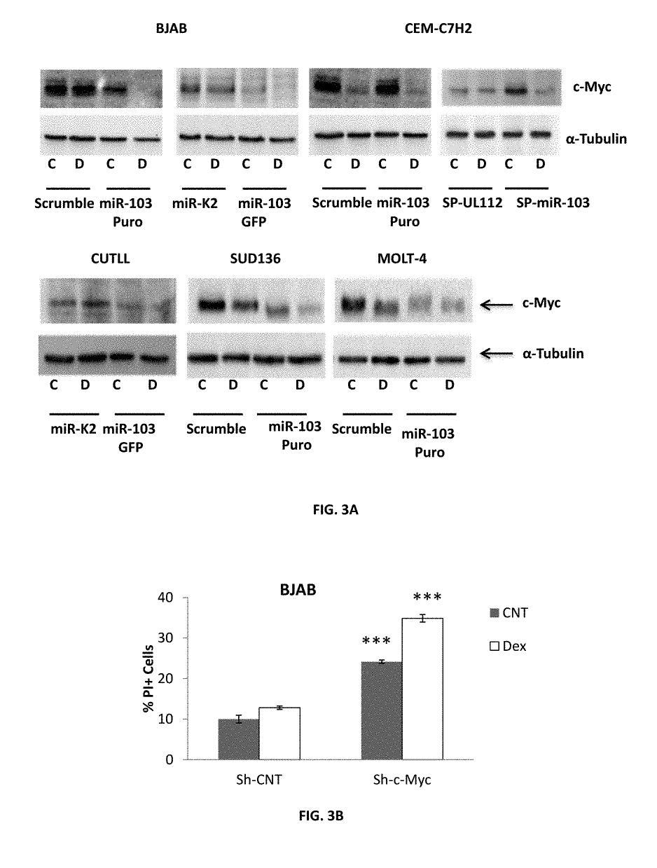

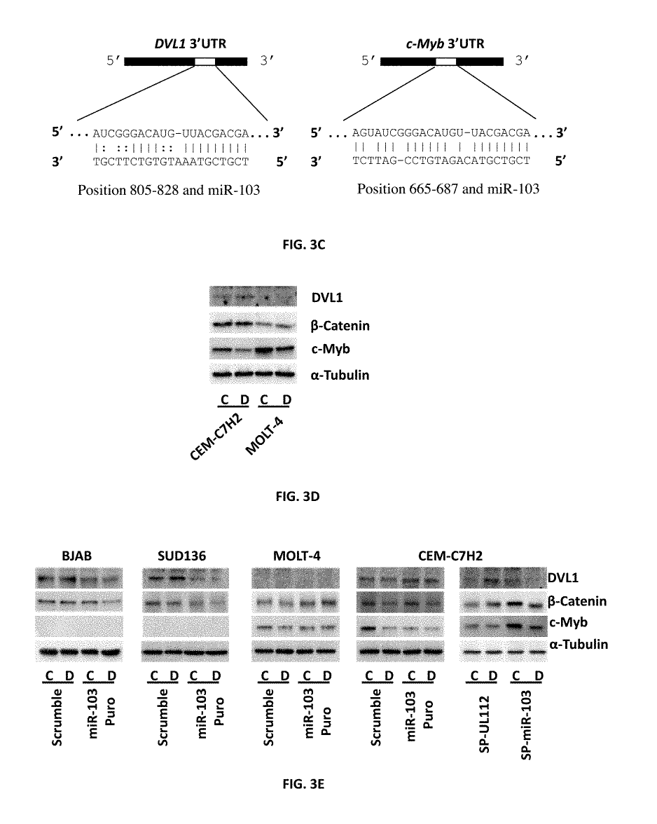

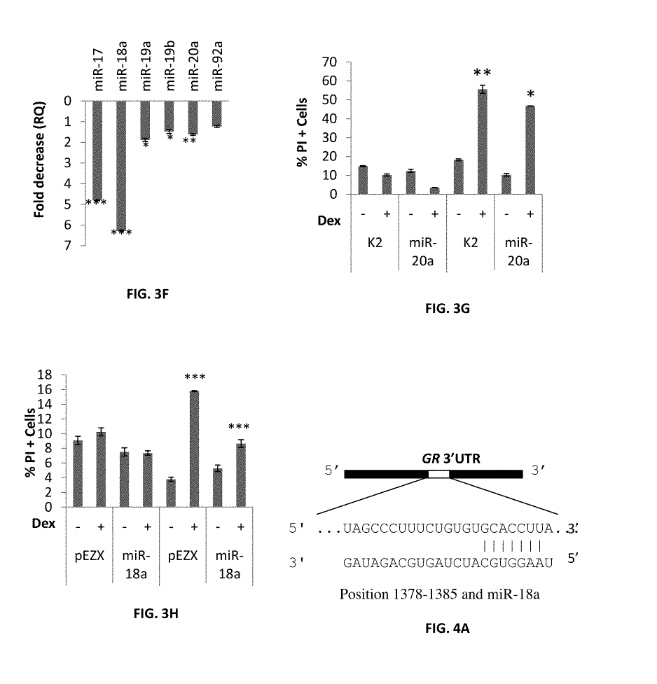

To clarify the mode by which miR-103 confers GCIA, miR-103 effect on consensus GCIA proteins was examined. c-Myc, which is downregulated by GC in sensitive CEM-C7H2 but not in resistant cells (FIG. 1D), was reduced by miR-103 overexpression and further downregulated following Dex treatment (FIGS. 3A and 2L). Moreover, sponging miR-103 upregulated c-Myc expression (FIG. 3A). However, upon Dex treatment, c-Myc expression was nullified (FIG. 3A), explaining why GCIA of miR-103 sponged cells was only decreased but not abolished (FIG. 2A-B). Dex treatment upregulates miR-103 to levels excessive of the sponge absorption capacity. In order to demonstrate the impact of c-Myc ablation on GCIA, BJAB cells were transfected with sh-RNA of c-Myc. This manipulation imitates the effect of miR-103 overexpression on GCIA (FIG. 3B).

### Example 5: miR-103 Downregulats c-Myc by Targeting Dv1-1 and c-Myb

c-Myc 3′UTR does not contain a seed sequence for miR-103, however miR-103 targets two c-Myc activators, c-Myb and DVL1 (FIG. 3C). DVL1 is known to activate β-Catenin, which similarly to c-Myb, acts as a transcription factor of c-Myc. c-Myb is highly expressed in both CEM-C7H2 and MOLT-4 cells, but undergoes significant ablation by Dex only in the former (FIG. 3D). The expression of DVL1 and β-Catenin was found high in CEM-C7H2, low in MOLT-4 and unaffected by Dex (FIG. 3D), indicating that Wnt signaling is unlikely to interfere with GCIA in T-ALL.

DVL1 and β-Catenin were downregulated by miR-103 in Burkitt's lymphoma cells, and upregulated when the level of miR-103 was reduced by its sponge (FIG. 3E). However, DVL1 was not detected in MOLT-4 and its expression in CEM-C7H2 was even upregulated when miR-103 was overexpressed (FIG. 3E). Likewise, the level of β-Catenin was unaffected by miR-103 in CEM-C7H2 and even upregulated in MOLT4 (FIG. 3E). By contrast, miR-103 downregulates c-Myb expression in CEM-C7H2 and MOLT-4 T-ALL cells (FIG. 3E). Moreover, upon miR-103 sponging, c-Myb expression is increased, but almost eliminated upon Dex treatment (FIG. 3E). c-Myb wasn't detected in B cell lymphomas, as this protein plays an essential role mostly in T, but less in B cell development.

### Example 6: miR-103 Downregulates Expression of miR-17˜92a

c-Myc is a transcription factor of miR-17˜92a 32. Indeed, in CEM-C7H2 cells, which responded to GC with downregulation of c-Myc (FIG. 1D), miR-17˜92a was also downregulated following Dex treatment (FIG. 2N). However, in resistant MOLT-4 cells, both miR-17˜92a and c-Myc were almost unaffected by Dex (FIG. 2O and FIG. 1D). When miR-103 was overexpressed in resistant BJAB cells, a reduced expression of miR-17˜92a was recorded (FIG. 1F). To verify which of the downregulated miR-17˜92a miRNAs are involved in miR-103 mediated GCIA, BJAB cells overexpressing miR103-were transfectedwith plasmids containing each miRNA of miR-17˜92a. Only miR-18a and miR20a re-expression counteracted the miR-103-mediated GCIA (FIGS. 3G-H and FIGS. S2P-S).

### Example 7: Suppression of miR-18a by miR-103 Enables GR Upregulation

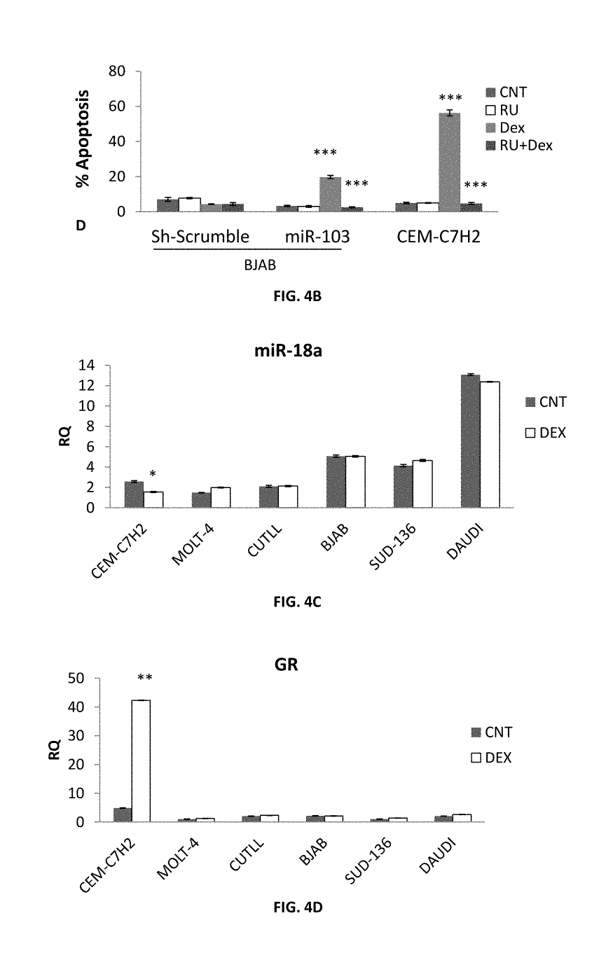

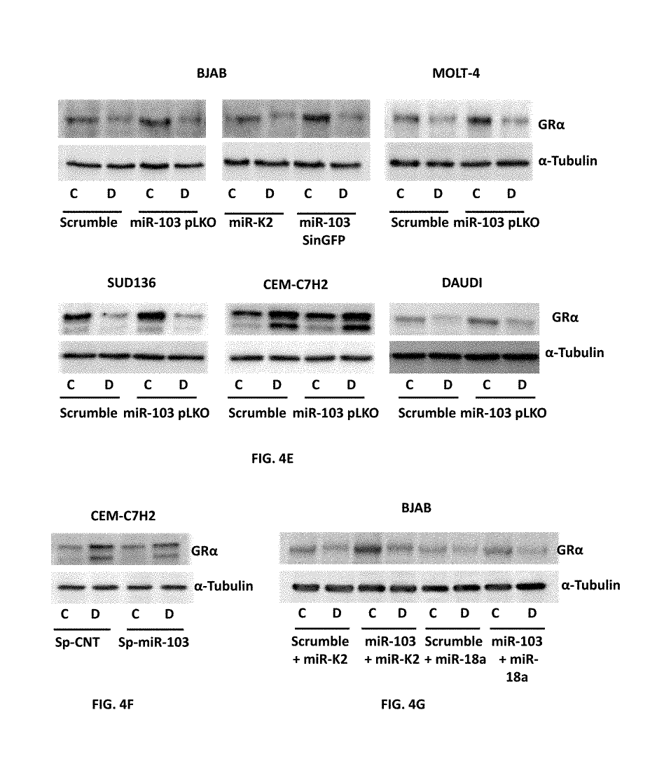

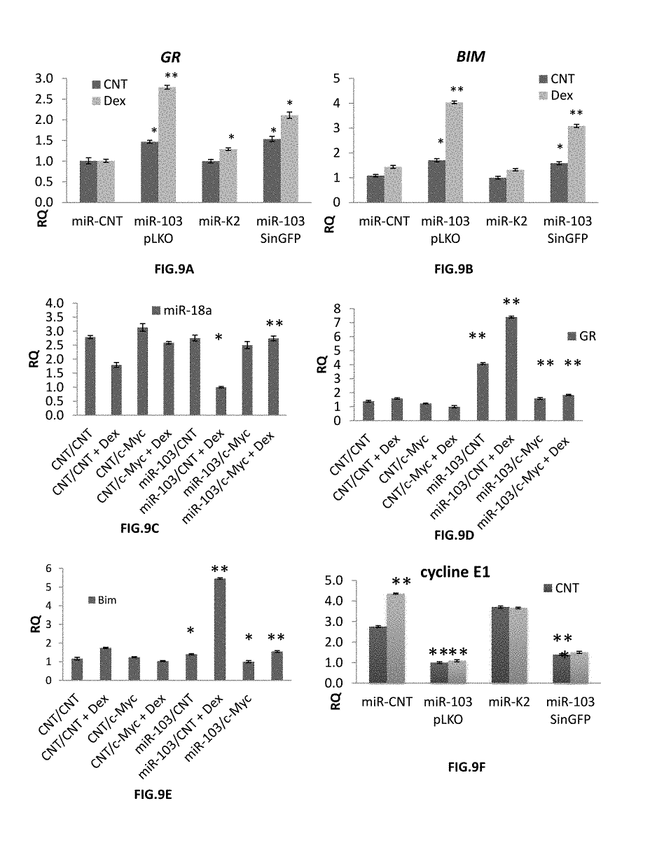

In order to decipher the mode by which miR-103-induced downregulation of miR-18a and miR-20a facilitates GCIA, Inventors further screened for target genes of miR-18a and miR-20a. Computational analysis revealed that miR-18a possesses a 8mer base pair sequence with the 3′UTR of GR mRNA (NR3C1) (FIG. 4A). It was previously reported that GR is upregulated in GC-sensitive cells but downregulated in GC-resistant cells upon GC treatment. By quantifying mRNA levels, GR was indeed found to be upregulated in GC-sensitive cells and unchanged in GC-resistant cells upon Dex treatment (FIG. 4D). These results suggest that GR downregulation in GC-resistant cells is regulated post-transcriptionally. Dex-induced GR mRNA upregulation was found to correlates with miR-18a downregulation (FIG. 4C-D), whereas when GR level was unaffected by Dex, the level of miR-18a also remained unchanged (FIG. 4C-D). In contrast to mRNA regulation, GR protein was downregulated by Dex in GC-resistant cells while upregulated in GC-sensitive CEM-C7H2 (FIG. 4E). miR-103 overexpression elicits both GR protein and mRNA upregulation (FIG. 4E and FIG. 9A). Furthermore, sponging of miR-103 reduced the Dex-induced GR upregulation (FIG. 4F), suggesting that GR is auto-upregulated, in part, by miR-103 upregulation. In contrast to GR mRNA, which was augmented following Dex treatment in miR-103 overexpressing cells, the miR-103-mediated upregulation of GR protein was reversed by Dex treatment, as noted in GC-resistant cells (FIG. 4E). However, although GR is finally downregulated by Dex (FIG. 4E), a substantial GCIA is still detectable (FIG. 2D-G).

To reinforce the surmise that miR-103 downregulates miR-18a and upregulates GR by decreasing the level of c-Myc, c-Myc was overexpressed in miR-103 transfected cells. Indeed, c-Myc overexpression reversed the effect of miR-103 on miR-18a and GR expression (FIGS. 9C-D). Finally, in order to confirm that miR-103-induced upregulation of GR is prompted by miR-18a downregulation, miR-18a expression was restored in miR-103 transfected cells. Restoring miR-18a expression in miR-103 transfected cells led to inhibition of the GR upregulation mediated by miR-103 (FIG. 4G). The data suggest that GCIA induced by miR-103 is mediated, in part, by downregulation of c-Myc, which is followed by miR-18a downregulation and GR upregulation. Indeed, inhibition of GR by RU486 prevented GCIA in GC-sensitive CEM-C7H2 or in GC-resistant BJAB cells sensitized by transfection with miR-103 (FIG. 4B).

### Example 8: Suppression of miR-20a by miR-103 Enables BIM Upregulation

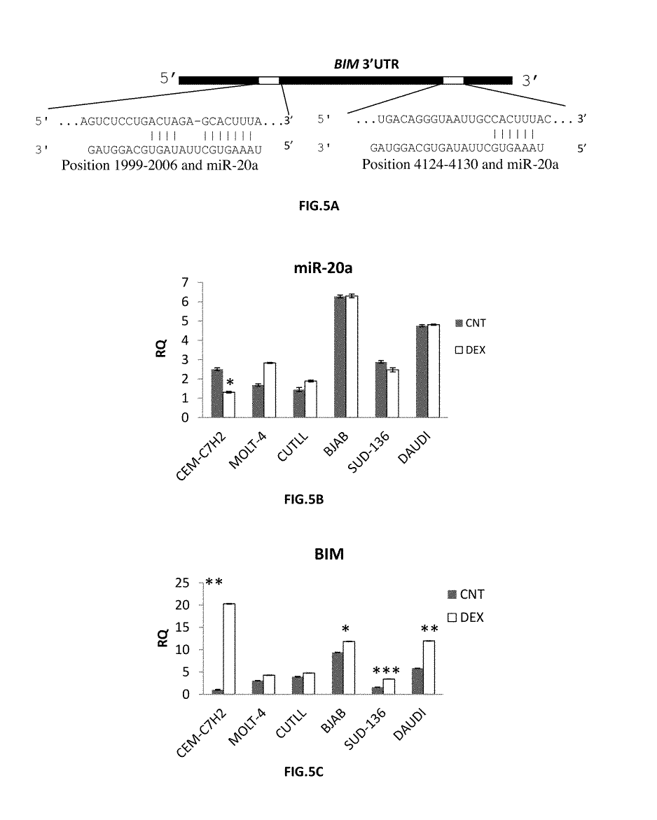

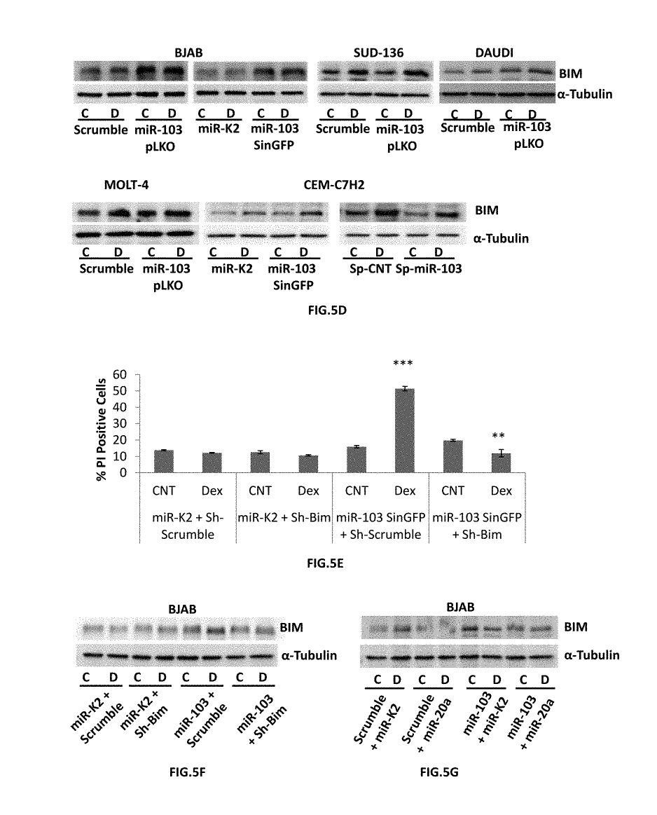

Another function of miR-103 is to downregulate miR-20a (FIG. 3F) that partakes in advancing GCIA (FIG. 3G). when searching for potential targets of miR-20a that are involved in GCIA, it was found that miR-20a has two conserved sites compatible with the 3′UTR of BIM mRNA (FIG. 5A). Indeed, BIM and miR-20a are reciprocally regulated in GC-sensitive cells: while BIM expression is upregulated by Dex (FIG. 5C) 8, miR-20a is concomitantly downregulated (FIG. 5B). Interestingly, although miR-20a did not change in Dex treated BJAB, SUD-136 and DAUDI cells (FIG. 5B), BIM was still upregulated significantly in DAUDI cells and slightly in the other GC-resistant cells (FIG. 5C), suggesting that Dex induces BIM upregulation by an additional mechanism.

To establish a link between miR-103-induced miR-20a downregulation and BIM upregulation, the effect of miR-103 overexpression on BIM, was examined. Indeed, miR-103 induced BIM upregulation both at the protein and mRNA levels, an activity that is augmented by Dex (FIG. 5D and FIG. 9B). Furthermore, sponging of miR-103 in CEM-C7H2 significantly reduced both basal and Dex-induced BIM levels (FIG. 5D). The role of BIM upregulation in miR-103 mediated GCIA was verified with sh-RNA studies. Downregulating BIM expression abrogates miR-103 sensitization to GCIA (FIG. 5E). Indeed, Sh-BIM assay brought BIM expression down to its basal level as in non-miR-103 transfected cells (FIG. 5F). In order to demonstrate that miR-103 upregulates BIM expression by decreasing the level of c-Myc, c-Myc was overexpressed in miR-103 transfected cells and reversal of the miR-103 effect on BIM expression was observed (FIG. 10E). miR-103-induced BIM upregulation was demonstrated to be effectuated via the downregulation of miR-20a. While the level of BIM is upregulated upon miR-103 transfection, it is re-suppressed when miR-20a expression is restored (FIG. 5G).

### Example 9: miR-103 Inhibits Cellular Proliferation

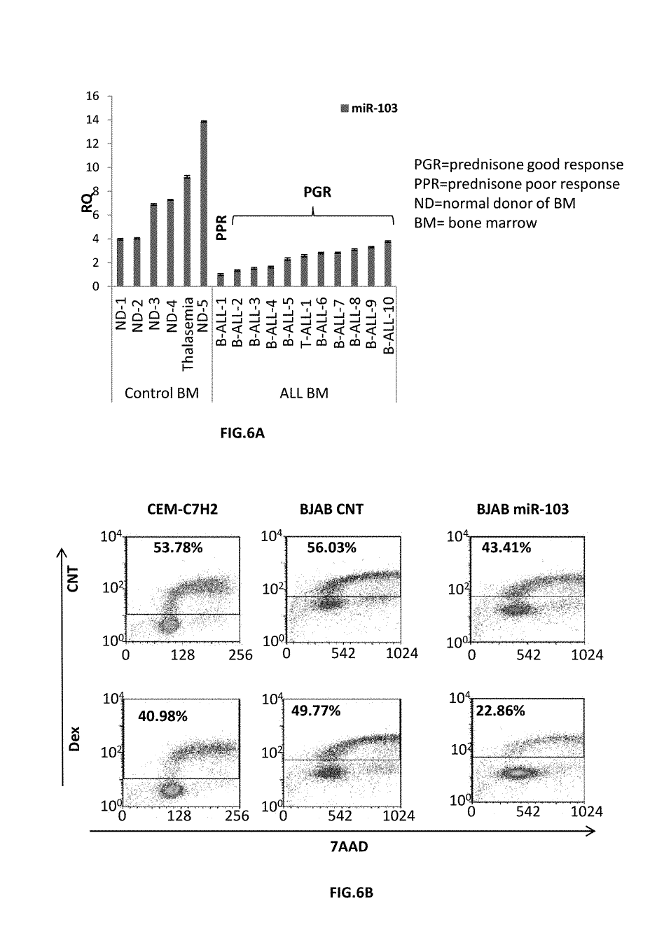

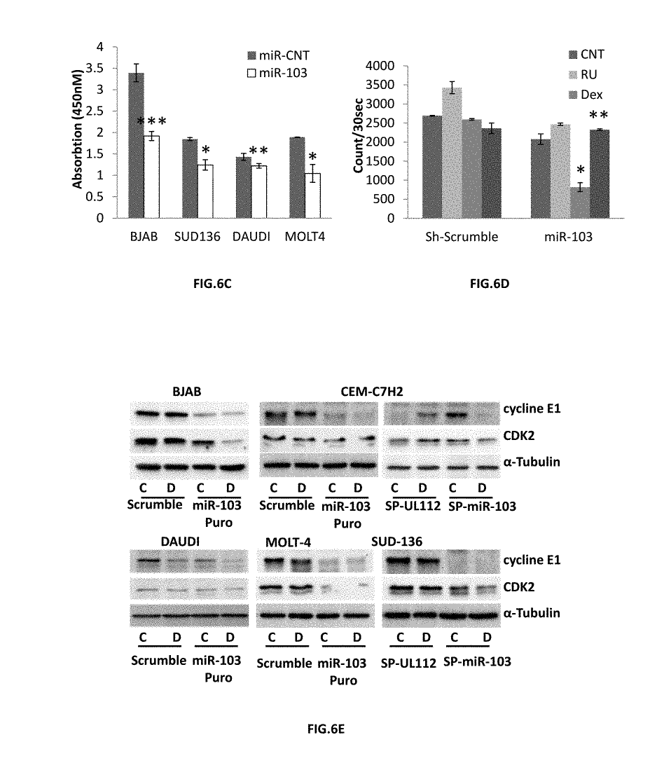

Basal expression of miR-103 in leukemia patients was evaluated as compared to healthy counterparts. To this end, level of miR-103 in bone marrow (BM) mononuclear cells from healthy donors and ALL pediatric patients was analyzed. The miR-103 level was significantly downregulated in ALL BM compared with normal BM (FIG. 6A). Interestingly, the lowest level of miR-103 was detected only in patient displaying prednisone poor response (PPR), while other patients displayed prednisone good response (PGR) (FIG. 6A). In addition to apoptosis, GC also induces cell cycle arrest 21, 45. Therefore a BRDU incorporation assay was performed and results demonstrate that miR-103 by itself significantly reduced cell proliferation (FIG. 6B-C). Addition of Dex augmented this effect in both GC-sensitive CEM-C7H2 and miR-103 overexpressing cells (FIG. 6B). miR-103 also reduced the total cell number in the culture, which was further decreased by Dex but abolished when GR was inhibited by RU486 (FIG. 6D). This observation indicates that miR-103 may act as a tumor suppressor by both conferring sensitivity to GCIA and inhibiting cellular proliferation. Indeed, cycline E1 and its positive regulator CDK2, which are essential for the G1->S transition, are direct targets of miR-103 46, and are downregulated by miR-103 overexpression in our study as well (FIG. 6E and FIGS. 10F-G). Vice versa, when miR-103 is sponged, there is a marked induction of both cycline E1 and CDK2 expression (FIG. 6E). Inhibition of cycline E1 and CDK2 by miR-103 is c-Myc independent.

### Example 10: A Model of miR-103 Network in GCIA

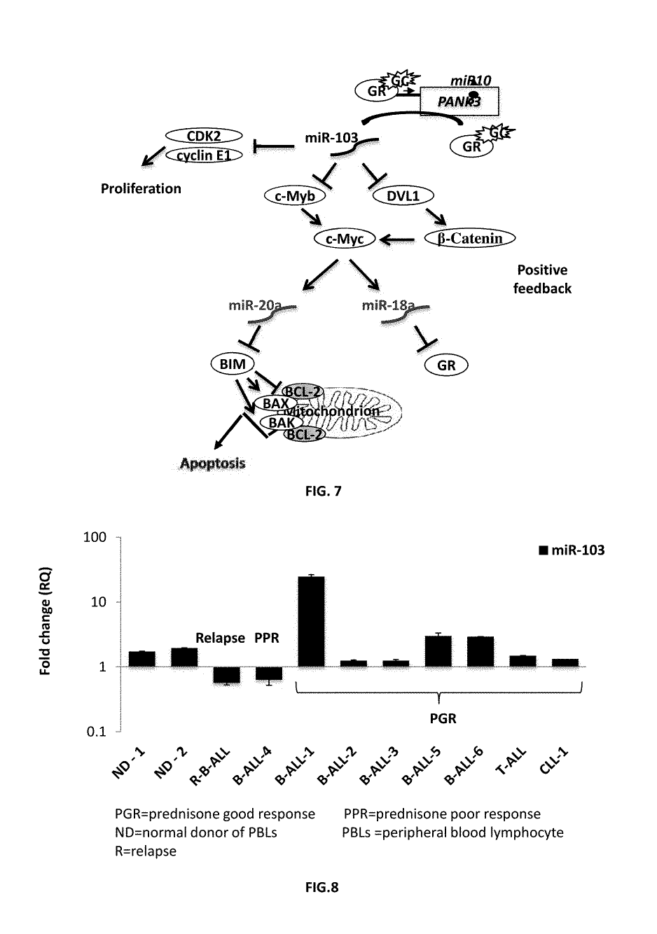

The described findings indicate that enforced miR-103 expression in GC-resistant cells conferred sensitivity to GC-induced apoptosis. Vice versa, cellular miR-103 inhibition reduced GC-induced apoptosis in GC-sensitive cells. miR-103 overexpression in GC-resistant cells imitates the cellular apoptotic pathway of GC occurring only in GC-sensitive cells by enforcing the upregulation of GR and Bim by downregulating c-Myc which is followed by miR-18a and miR-20a decrease. Downregulation of miR-18a mediates GR upregulation whereas downregulation of miR-20a mediates Bim upregulation. These data suggest that miR-103 to be a marker that predicts the response of lymphoma and leukemia to GC. Furthermore, treatment that enforces miR-103 upregulation would confer GC-induced apoptosis on otherwise GC-resistant leukemias and lymphomas; the signaling network is illustrated in FIG. 7.

### Example 11: The Expression of miR-103 in Bone Marrow and Blood Correlates to Patients Status

The outline of the study was to collect blood samples from acute lymphoblastic leukemia (ALL) patients at diagnosis, incubate the blood with or without PRED overnight and then measure miR-103 expression levels. 11 BMs of ALL patients and 6 BMs of normal pediatric donors were analyzed. Furthermore, peripheral blood samples from 8 pediatric ALL patients and 1 chronic lymphoblastic leukemia (CLL) patient were collected. The CLL and 5 ALL samples were treated with PRED in vitro while from the additional 3 ALL patients blood was taken at diagnosis and at day 1 following PRED treatment. All the samples were processed by qRT-PCR to determine the level of intra-cellular miR-103. Peripheral blood mononuclear cells (PBMC) were isolated, RNA was extracted and subjected to qRT-PCR. This study revealed that miR-103 is upregulated by PRED in peripheral malignant lymphoblasts of PRED good responders (PGR) ALL patients (similar to normal lymphocytes), while it is downregulated in prednisone poor response (PPR) ALL patients (FIG. 8). To improve this assay, blood was collected at diagnosis and at day 1 post PRED therapy, and a more significant miR-103 modulation was observed (FIG. 8 B-cell ALL (B-ALL)-1, 5-6). These findings together with the findings that PPR patients have the lowest miR-103 levels and miR-103 expression is significantly lower in ALL patients than in healthy children (FIG. 6A), indicate that basal expression of miR-103 partakes in determining the PRED response outcome and that ALL development is accompanied by miR-103 downregulation. These experiments further indicate that evaluating miR-103 expression in ALL cells prior and immediately after commencement of PRED therapy can provide valuable indication regarding the usefulness of applying PRED as a first choice in newly diagnosed patients. The data further highlights miR-103 as a good biomarker predicting leukemia response to PRED therapy. Hence, detection of miR-103 in leukemic cells may become a prognostic assay that facilitates personalized therapy of leukemia patients with PRED.

### Example 12: Development of a Single Cells Flow Cytometry Assay for Detection of miR-103

In order to improve the mode of miR-103 detection a novel assay based on staining with miR-103 fluorescinated probe and flow cytometry analysis is provided. This assay offers the ability to measure miR-103 at a single cell level, as well as an option for combining additional cellular markers in a single assay. In addition this assay offers shorter time of data acquisition as compared to real time PCR.

ALL lymphoblasts are of heterogenic nature; by using a flow cytometry assay small proportion of PPR cells within a large PGR ALL population can be detected. Identification of such cells should predict poor response even if the blast count at day 8 is lower than 1000/mL, thus enabling re-evaluation of a decision between high risk (HR) and non-HR regimen. The percentage of ALL blast in the whole peripheral blood lymphocytes (PBLs) may vary between 25%-99%. A flow cytometry analysis can distinguish between such cells by staining with specific cellular markers (CD20 for B-ALL and CD3 for T-ALL). Finally, a short assay with a definitive outcome makes flow cytometry a method of choice in measuring miR-103 levels.

1. A method for predicting responsiveness of a subject to glucocorticoids (GC) treatment, the method comprising determining the expression level of one or more miRNAs selected from the group consisting of: miR-103, miR-30e, miR-30d, miR-181a*, miR-15b* and miR-21 in a sample obtained from the subject, wherein modulation of expression levels of said one or more miRNAs compared to control indicates that the subject will be responsive to GC treatment.

2. The method of claim 1, wherein responsiveness of the subject to GC treatment is indicated by at least one of: increased expression levels of miR-103 compared to control; and modulation of expression of a plurality of miRNAs selected from the group consisting of: miR-103, miR-30e, miR-30d, miR-181a*, miR-15b* and miR-21.

3. (canceled)

4. The method of claim 1, further comprising determining the expression level of at least one miRNA selected from the group consisting of: miR-15b, miR-16, miR-181a, has-let-7f, miR-186, miR-223, miR-103*, miR-20a, miR-92a, miR-17, miR-30e*, miR-19b, miR-18a, miR-19a, wherein at least one of: increased expression of one or more miRNAs selected from the group consisting of: miR-103, miR-30e, miR-30d, miR-15b*, miR-21, miR-15b, miR-16, miR-181a, let-7f, miR-186 and miR-223, compared to control; and decreased expression of one or more miRNAs selected from the group consisting of: miR-181a*, miR-103*, miR-20a, miR-92a, miR-17, miR30e*, miR-19b, miR-18a and miR-19a, compared to control indicates that the subject will be responsive to GC treatment.

5. The method of claim 1, for treating a subject with GC, the method further comprises the step of:
administering a therapeutically effective amount of GC to the susceptible subject thereby treating said subject with GC.

6. (canceled)

7. (canceled)

8. (canceled)

9. (canceled)

10. (canceled)

11. The method of claim 1, wherein said subject is afflicted with a disease typically treated with GCs, optionally wherein said disease is cancer.

12. (canceled)

13. The method of claim 11, wherein said cancer is selected from the group consisting of: a hematopoietic cancer, osteosarcoma or small cell lung cancer.

14. (canceled)

15. The method of claim 1, wherein said sample is selected from the group consisting of: blood, plasma and serum.

16. The method of claim 1, wherein said sample is peripheral blood lymphoblasts (PBLs).

17. A kit comprising reagents adapted to specifically determine the expression level of one or more miRNAs selected from the group consisting of: miR-103, miR-30e, miR-30d, miR-181a*, miR-15b* and miR-21.

18. The kit of claim 17, useful for predicting susceptibility of a subject to GC therapy.

19. The kit of claim 17, comprising reagents adapted to determine the expression level of miR-103.

20. The kit of claim 17, further comprising reagents adapted to determine the expression level of at least one additional miRNA selected from the group consisting of: miR-15b, miR-16, miR-181a, let-7f, miR-186, miR-223, miR-103*, miR-20a, miR-92a, miR-17, miR-30e*, miR-19b, miR-18a and miR-19a.

21. The kit of claim 17, wherein said reagents are selected from miRNA hybridization or amplification reagents, and one or more miRNAs-specific probe or amplification primer.

22. The kit of claim 17, further comprising means for obtaining a blood sample.

23. (canceled)

24. A method for sensitizing a subject in need thereof to GC therapy, the method comprising administering to the subject a pharmaceutical composition comprising one or more miRNAs selected from the group consisting of: miR-103, miR-30e, miR-30d, miR-15b* and miR-21, and a pharmaceutically acceptable carrier.

25. The method of claim 24, further comprising one or more miRNAs selected from the group consisting of miR-15b, miR-16, miR-181a, let-7f, miR-186 and miR-223.

26. The method of claim 24, further comprising an miRNA inhibitor of one or more miRNAs selected from the group consisting of miR-103*, miR-181a*, miR-20a, miR-92a, miR-17, miR30e*, miR-19b, miR-18a and miR-19a.

27. The method of claim 24, wherein said subject is afflicted with a disease typically treated with GCs.

28. The method of claim 24, wherein said subject is afflicted with a cancer typically treated with GCs.

29. (canceled)

30. (canceled)

31. The method of claim 24, wherein said subject is resistant to GCs therapy.


SEQUENCE LISTING (60 sequences)

SEQ ID NO: 1 - RNA, 23 nt, Homo sapiens
agcagcauuguacagggcuauga

SEQ ID NO: 2 - RNA, 22 nt, Homo sapiens
uguaaacauccuugacuggaag

SEQ ID NO: 3 - RNA, 22 nt, Homo sapiens
uguaaacauccccgacuggaag

SEQ ID NO: 4 - RNA, 22 nt, Homo sapiens
uagcagcacaucaugguuuaca

SEQ ID NO: 5 - RNA, 22 nt, Homo sapiens
uagcagcacguaaauauuggcg

SEQ ID NO: 6 - RNA, 22 nt, Homo sapiens
accaucgaccguugauuguacc

SEQ ID NO: 7 - RNA, 22 nt, Homo sapiens
cgaaucauuauuugcugcucua

SEQ ID NO: 8 - RNA, 22 nt, Homo sapiens
uagcuuaucagacugauguuga

SEQ ID NO: 9 - RNA, 23 nt, Homo sapiens
aacauucaacgcugucggugagu

SEQ ID NO: 10 - RNA, 22 nt, Homo sapiens
ugagguaguagauuguauaguu

SEQ ID NO: 11 - RNA, 22 nt, Homo sapiens
caaagaauucuccuuuugggcu

SEQ ID NO: 12 - RNA, 22 nt, Homo sapiens
cguguauuugacaagcugaguu

SEQ ID NO: 13 - RNA, 23 nt, Homo sapiens
ucauagcccuguacaaugcugcu

SEQ ID NO: 14 - RNA, 23 nt, Homo sapiens
uaaagugcuuauagugcagguag

SEQ ID NO: 15 - RNA, 23 nt, Homo sapiens
agguugggaucgguugcaaugcu

SEQ ID NO: 16 - RNA, 23 nt, Homo sapiens
caaagugcuuacagugcagguag

SEQ ID NO: 17 - RNA, 22 nt, Homo sapiens
cuuucagucggauguuuacagc

SEQ ID NO: 18 - RNA, 23 nt, Homo sapiens
aguuuugcagguuugcauccagc

SEQ ID NO: 19 - RNA, 23 nt, Homo sapiens
uaaggugcaucuagugcagauag

SEQ ID NO: 20 - RNA, 22 nt, Homo sapiens
aguuuugcauaguugcacuaca

SEQ ID NO: 21 - RNA, 78 nt, Homo sapiens
uacugcccucggcuucuuuacagugcugccuuguugcauauggaucaagcagcauuguacagggcuaugaaggcauug

SEQ ID NO: 22 - RNA, 92 nt, Homo sapiens
gggcagucuuugcuacuguaaacauccuugacuggaagcuguaagguguucagaggagcuuucagucggauguuuacagcggcaggcugcca

SEQ ID NO: 23 - RNA, 70 nt, Homo sapiens
guuguuguaaacauccccgacuggaagcuguaagacacagcuaagcuuucagucagauguuugcugcuac

SEQ ID NO: 24 - RNA, 98 nt, Homo sapiens
uugaggccuuaaaguacuguagcagcacaucaugguuuacaugcuacagucaagaugcgaaucauuauuugcugcucuagaaauuuaaggaaauucau

SEQ ID NO: 25 - RNA, 89 nt, Homo sapiens
gucagcagugccuuagcagcacguaaauauuggcguuaagauucuaaaauuaucuccaguauuaacugugcugcugaaguaagguugac

SEQ ID NO: 26 - RNA, 110 nt, Homo sapiens
agaagggcuaucaggccagccuucagaggacuccaaggaacauucaacgcugucggugaguuugggauuugaaaaaaccacugaccguugacuguaccuugggguccuua

SEQ ID NO: 27 - RNA, 98 nt, Homo sapiens; identical to SEQ ID NO: 24

SEQ ID NO: 28 - RNA, 72 nt, Homo sapiens
ugucggguagcuuaucagacugauguugacuguugaaucucauggcaacaccagucgaugggcugucugaca

SEQ ID NO: 29 - RNA, 110 nt, Homo sapiens; identical to SEQ ID NO: 26

SEQ ID NO: 30 - RNA, 87 nt, Homo sapiens
ucagagugagguaguagauuguauaguugugggguagugauuuuacccuguucaggagauaacuauacaaucuauugccuucccuga

SEQ ID NO: 31 - RNA, 86 nt, Homo sapiens
ugcuuguaacuuuccaaagaauucuccuuuugggcuuucugguuuuauuuuaagcccaaaggugaauuuuuugggaaguuugagcu

SEQ ID NO: 32 - RNA, 110 nt, Homo sapiens
ccuggccuccugcagugccacgcuccguguauuugacaagcugaguuggacacuccaugugguagagugucaguuugucaaauaccccaagugcggcacaugcuuaccag

SEQ ID NO: 33 - RNA, 62 nt, Homo sapiens
ucauagcccuguacaaugcugcuugauccauaugcaacaaggcagcacuguaaagaagccga

SEQ ID NO: 34 - RNA, 71 nt, Homo sapiens
guagcacuaaagugcuuauagugcagguaguguuuaguuaucuacugcauuaugagcacuuaaaguacugc

SEQ ID NO: 35 - RNA, 78 nt, Homo sapiens
cuuucuacacagguugggaucgguugcaaugcuguguuucuguaugguauugcacuugucccggccuguugaguuugg

SEQ ID NO: 36 - RNA, 84 nt, Homo sapiens
gucagaauaaugucaaagugcuuacagugcagguagugauaugugcaucuacugcagugaaggcacuuguagcauuauggugac

SEQ ID NO: 37 - RNA, 92 nt, Homo sapiens; identical to SEQ ID NO: 22

SEQ ID NO: 38 - RNA, 87 nt, Homo sapiens
cacuguucuaugguuaguuuugcagguuugcauccagcugugugauauucugcugugcaaauccaugcaaaacugacugugguagug

SEQ ID NO: 39 - RNA, 71 nt, Homo sapiens
uguucuaaggugcaucuagugcagauagugaaguagauuagcaucuacugcccuaagugcuccuucuggca

SEQ ID NO: 40 - RNA, 82 nt, Homo sapiens
gcaguccucuguuaguuuugcauaguugcacuacaagaagaauguaguugugcaaaucuaugcaaaacugaugguggccugc

SEQ ID NO: 41 - DNA, 72 bp, Artificial sequence
  Synthetic
gatccccagcagcattgtacagggctatgattcaagagatcatagccctgtacaatgctgcttttttggaaa

SEQ ID NO: 42 - DNA, 72 bp, Artificial sequence
  Synthetic
agcttttccaaaaaagcagcattgtacagggctatgatctcttgaatcatagccctgtacaatgctgctggg

SEQ ID NO: 43 - DNA, 70 bp, Artificial sequence
  Synthetic
gatcccctgtaaacatccttgactggaagttcaagagacttccagtcaaggatgtttacatttttggaaa

SEQ ID NO: 44 - DNA, 70 bp, Artificial sequence
  Synthetic
agcttttccaaaaatgtaaacatccttgactggaagtctcttgaacttccagtcaaggatgtttacaggg

SEQ ID NO: 45 - DNA, 70 bp, Artificial sequence
  Synthetic
gatcccctgtaaacatccccgactggaagttcaagagacttccagtcggggatgtttacatttttggaaa

SEQ ID NO: 46 - DNA, 70 bp, Artificial sequence
  Synthetic
agcttttccaaaaatgtaaacatccccgactggaagtctcttgaacttccagtcggggatgtttacaggg

SEQ ID NO: 47 - DNA, 72 bp, Artificial sequence
  Synthetic
gatcccctgtgcaaatccatgcaaaactgattcaagagatcagttttgcatggatttgcacatttttggaaa

SEQ ID NO: 48 - DNA, 72 bp, Artificial sequence
  Synthetic
agcttttccaaaaatgtgcaaatccatgcaaaactgatctcttgaatcagttttgcatggatttgcacaggg

SEQ ID NO: 49 - DNA, 72 bp, Artificial sequence
  Synthetic
gatcccctaaagtgcttatagtgcaggtagttcaagagactacctgcactataagcactttatttttggaaa

SEQ ID NO: 50 - DNA, 72 bp, Artificial sequence
  Synthetic
agcttttccaaaaataaagtgcttatagtgcaggtagtctcttgaactacctgcactataagcactttaggg

SEQ ID NO: 51 - DNA, 70 bp, Artificial sequence
  Synthetic
gatcccctgtcagtttgtcaaataccccattcaagagatggggtatttgacaaactgacatttttggaaa

SEQ ID NO: 52 - DNA, 70 bp, Artificial sequence
  Synthetic
agcttttccaaaaatgtcagtttgtcaaataccccatctcttgaatggggtatttgacaaactgacaggg

SEQ ID NO: 53 - DNA, 70 bp, Artificial sequence
  Synthetic
gatcccccgaatcattatttgctgctctattcaagagatagagcagcaaataatgattcgtttttggaaa

SEQ ID NO: 54 - DNA, 70 bp, Artificial sequence
  Synthetic
agcttttccaaaaacgaatcattatttgctgctctatctcttgaatagagcagcaaataatgattcgggg

SEQ ID NO: 55 - DNA, 70 bp, Artificial sequence
  Synthetic
gatcccctagcagcacgtaaatattggcgttcaagagacgccaatatttacgtgctgctatttttggaaa

SEQ ID NO: 56 - DNA, 70 bp, Artificial sequence
  Synthetic
agcttttccaaaaatagcagcacgtaaatattggcgtctcttgaacgccaatatttacgtgctgctaggg

SEQ ID NO: 57 - DNA, 80 bp, Artificial sequence
  Synthetic
tcgactcatagccctgtgtatgctgctagagtcatagccctgtgtatgctgctagagtcatagccctgtgtatgctgcta

SEQ ID NO: 58 - DNA, 80 bp, Artificial sequence
  Synthetic
agcttagcagcatacacagggctatgactctagcagcatacacagggctatgactctagcagcatacacagggctatgag

SEQ ID NO: 59 - DNA, 80 bp, Artificial sequence
  Synthetic
agctttcatagccctgtgtatgctgctagagtcatagccctgtgtatgctgctagagtcatagccctgtgtatgctgctg

SEQ ID NO: 60 - DNA, 80 bp, Artificial Sequence
  Synthetic
aattcagcagcatacacagggctatgactctagcagcatacacagggctatgactctagcagcatacacagggctatgaa